SEQUENCE LISTING (96 sequences)

SEQ ID NO: 1 - DNA, 50 bp, Homo sapiens
caaccgccttttcatcaatcgcccacatcactcgagacgtaaattatggc

SEQ ID NO: 2 - DNA, 50 bp, Homo sapiens
caaccgccttatcatcaatcgcccacatcactcgagacgtaaattatggc

SEQ ID NO: 3 - DNA, 50 bp, Homo sapiens
caaccgccttctcatcaatcgcccacatcactcgagacgtaaattatggc

SEQ ID NO: 4 - DNA, 50 bp, Homo sapiens
caaccgccttgtcatcaatcgcccacatcactcgagacgtaaattatggc

SEQ ID NO: 5 - DNA, 50 bp, Homo sapiens
caaccgcctttacatcaatcgcccacatcactcgagacgtaaattatggc

SEQ ID NO: 6 - DNA, 50 bp, Homo sapiens
caaccgcctttccatcaatcgcccacatcactcgagacgtaaattatggc

SEQ ID NO: 7 - DNA, 50 bp, Homo sapiens
caaccgcctttgcatcaatcgcccacatcactcgagacgtaaattatggc

SEQ ID NO: 8 - DNA, 50 bp, Homo sapiens
caaccgcctttttatcaatcgcccacatcactcgagacgtaaattatggc

SEQ ID NO: 9 - DNA, 50 bp, Homo sapiens
caaccgccttttaatcaatcgcccacatcactcgagacgtaaattatggc

SEQ ID NO: 10 - DNA, 50 bp, Homo sapiens
caaccgccttttgatcaatcgcccacatcactcgagacgtaaattatggc

SEQ ID NO: 11 - DNA, 50 bp, Homo sapiens
caaccgccttttcttcaatcgcccacatcactcgagacgtaaattatggc

SEQ ID NO: 12 - DNA, 50 bp, Homo sapiens
caaccgccttttcctcaatcgcccacatcactcgagacgtaaattatggc

SEQ ID NO: 13 - DNA, 50 bp, Homo sapiens
caaccgccttttcgtcaatcgcccacatcactcgagacgtaaattatggc

SEQ ID NO: 14 - DNA, 50 bp, Homo sapiens
caaccgccttttcaacaatcgcccacatcactcgagacgtaaattatggc

SEQ ID NO: 15 - DNA, 50 bp, Homo sapiens
caaccgccttttcaccaatcgcccacatcactcgagacgtaaattatggc

SEQ ID NO: 16 - DNA, 50 bp, Homo sapiens
caaccgccttttcagcaatcgcccacatcactcgagacgtaaattatggc

SEQ ID NO: 17 - DNA, 50 bp, Homo sapiens
caaccgccttttcattaatcgcccacatcactcgagacgtaaattatggc

SEQ ID NO: 18 - DNA, 50 bp, Homo sapiens
caaccgccttttcataaatcgcccacatcactcgagacgtaaattatggc

SEQ ID NO: 19 - DNA, 50 bp, Homo sapiens
caaccgccttttcatgaatcgcccacatcactcgagacgtaaattatggc

SEQ ID NO: 20 - DNA, 50 bp, Homo sapiens
caaccgccttttcatctatcgcccacatcactcgagacgtaaattatggc

SEQ ID NO: 21 - DNA, 50 bp, Homo sapiens
caaccgccttttcatccatcgcccacatcactcgagacgtaaattatggc

SEQ ID NO: 22 - DNA, 50 bp, Homo sapiens
caaccgccttttcatcgatcgcccacatcactcgagacgtaaattatggc

SEQ ID NO: 23 - DNA, 50 bp, Homo sapiens
caaccgccttttcatcattcgcccacatcactcgagacgtaaattatggc

SEQ ID NO: 24 - DNA, 50 bp, Homo sapiens
caaccgccttttcatcactcgcccacatcactcgagacgtaaattatggc

SEQ ID NO: 25 - DNA, 50 bp, Homo sapiens
caaccgccttttcatcagtcgcccacatcactcgagacgtaaattatggc

SEQ ID NO: 26 - DNA, 50 bp, Homo sapiens
caaccgccttttcatcaaacgcccacatcactcgagacgtaaattatggc

SEQ ID NO: 27 - DNA, 50 bp, Homo sapiens
caaccgccttttcatcaaccgcccacatcactcgagacgtaaattatggc

SEQ ID NO: 28 - DNA, 50 bp, Homo sapiens
caaccgccttttcatcaagcgcccacatcactcgagacgtaaattatggc

SEQ ID NO: 29 - DNA, 50 bp, Homo sapiens
caaccgccttttcatcaattgcccacatcactcgagacgtaaattatggc

SEQ ID NO: 30 - DNA, 50 bp, Homo sapiens
caaccgccttttcatcaatagcccacatcactcgagacgtaaattatggc

SEQ ID NO: 31 - DNA, 50 bp, Homo sapiens
caaccgccttttcatcaatggcccacatcactcgagacgtaaattatggc

SEQ ID NO: 32 - DNA, 50 bp, Homo sapiens
caaccgccttttcatcaatcccccacatcactcgagacgtaaattatggc

SEQ ID NO: 33 - DNA, 50 bp, Homo sapiens
caaccgccttttcatcaatctcccacatcactcgagacgtaaattatggc

SEQ ID NO: 34 - DNA, 50 bp, Homo sapiens
caaccgccttttcatcaatcacccacatcactcgagacgtaaattatggc

SEQ ID NO: 35 - DNA, 50 bp, Homo sapiens
caaccgccttttcatcaatcgtccacatcactcgagacgtaaattatggc

SEQ ID NO: 36 - DNA, 50 bp, Homo sapiens
caaccgccttttcatcaatcgaccacatcactcgagacgtaaattatggc

SEQ ID NO: 37 - DNA, 50 bp, Homo sapiens
caaccgccttttcatcaatcggccacatcactcgagacgtaaattatggc

SEQ ID NO: 38 - DNA, 50 bp, Homo sapiens
caaccgccttttcatcaatcgctcacatcactcgagacgtaaattatggc

SEQ ID NO: 39 - DNA, 50 bp, Homo sapiens
caaccgccttttcatcaatcgcacacatcactcgagacgtaaattatggc

SEQ ID NO: 40 - DNA, 50 bp, Homo sapiens
caaccgccttttcatcaatcgcgcacatcactcgagacgtaaattatggc

SEQ ID NO: 41 - DNA, 50 bp, Homo sapiens
caaccgccttttcatcaatcgcctacatcactcgagacgtaaattatggc

SEQ ID NO: 42 - DNA, 50 bp, Homo sapiens
caaccgccttttcatcaatcgccaacatcactcgagacgtaaattatggc

SEQ ID NO: 43 - DNA, 50 bp, Homo sapiens
caaccgccttttcatcaatcgccgacatcactcgagacgtaaattatggc

SEQ ID NO: 44 - DNA, 50 bp, Homo sapiens
caaccgccttttcatcaatcgccctcatcactcgagacgtaaattatggc

SEQ ID NO: 45 - DNA, 50 bp, Homo sapiens
caaccgccttttcatcaatcgcccccatcactcgagacgtaaattatggc

SEQ ID NO: 46 - DNA, 50 bp, Homo sapiens
caaccgccttttcatcaatcgcccgcatcactcgagacgtaaattatggc

SEQ ID NO: 47 - DNA, 50 bp, Homo sapiens
caaccgccttttcatcaatcgcccatatcactcgagacgtaaattatggc

SEQ ID NO: 48 - DNA, 50 bp, Homo sapiens
caaccgccttttcatcaatcgcccaaatcactcgagacgtaaattatggc

SEQ ID NO: 49 - DNA, 50 bp, Homo sapiens
caaccgccttttcatcaatcgcccagatcactcgagacgtaaattatggc

SEQ ID NO: 50 - DNA, 50 bp, Homo sapiens
caaccgccttttcatcaatcgcccacttcactcgagacgtaaattatggc

SEQ ID NO: 51 - DNA, 50 bp, Homo sapiens
caaccgccttttcatcaatcgcccacctcactcgagacgtaaattatggc

SEQ ID NO: 52 - DNA, 50 bp, Homo sapiens
caaccgccttttcatcaatcgcccacgtcactcgagacgtaaattatggc

SEQ ID NO: 53 - DNA, 50 bp, Homo sapiens
caaccgccttttcatcaatcgcccacaacactcgagacgtaaattatggc

SEQ ID NO: 54 - DNA, 50 bp, Homo sapiens
caaccgccttttcatcaatcgcccacaccactcgagacgtaaattatggc

SEQ ID NO: 55 - DNA, 50 bp, Homo sapiens
caaccgccttttcatcaatcgcccacagcactcgagacgtaaattatggc

SEQ ID NO: 56 - DNA, 50 bp, Homo sapiens
caaccgccttttcatcaatcgcccacattactcgagacgtaaattatggc

SEQ ID NO: 57 - DNA, 50 bp, Homo sapiens
caaccgccttttcatcaatcgcccacataactcgagacgtaaattatggc

SEQ ID NO: 58 - DNA, 50 bp, Homo sapiens
caaccgccttttcatcaatcgcccacatgactcgagacgtaaattatggc

SEQ ID NO: 59 - DNA, 50 bp, Homo sapiens
caaccgccttttcatcaatcgcccacatctctcgagacgtaaattatggc

SEQ ID NO: 60 - DNA, 50 bp, Homo sapiens
caaccgccttttcatcaatcgcccacatccctcgagacgtaaattatggc

SEQ ID NO: 61 - DNA, 50 bp, Homo sapiens
caaccgccttttcatcaatcgcccacatcgctcgagacgtaaattatggc

SEQ ID NO: 62 - DNA, 50 bp, Homo sapiens
caaccgccttttcatcaatcgcccacatcattcgagacgtaaattatggc

SEQ ID NO: 63 - DNA, 50 bp, Homo sapiens
caaccgccttttcatcaatcgcccacatcaatcgagacgtaaattatggc

SEQ ID NO: 64 - DNA, 50 bp, Homo sapiens
caaccgccttttcatcaatcgcccacatcagtcgagacgtaaattatggc

SEQ ID NO: 65 - DNA, 50 bp, Homo sapiens
caaccgccttttcatcaatcgcccacatcacacgagacgtaaattatggc

SEQ ID NO: 66 - DNA, 50 bp, Homo sapiens
caaccgccttttcatcaatcgcccacatcacccgagacgtaaattatggc

SEQ ID NO: 67 - DNA, 50 bp, Homo sapiens
caaccgccttttcatcaatcgcccacatcacgcgagacgtaaattatggc

SEQ ID NO: 68 - DNA, 50 bp, Homo sapiens
caaccgccttttcatcaatcgcccacatcacttgagacgtaaattatggc

SEQ ID NO: 69 - DNA, 50 bp, Homo sapiens
caaccgccttttcatcaatcgcccacatcactagagacgtaaattatggc

SEQ ID NO: 70 - DNA, 50 bp, Homo sapiens
caaccgccttttcatcaatcgcccacatcactggagacgtaaattatggc

SEQ ID NO: 71 - DNA, 50 bp, Homo sapiens
caaccgccttttcatcaatcgcccacatcactctagacgtaaattatggc

SEQ ID NO: 72 - DNA, 50 bp, Homo sapiens
caaccgccttttcatcaatcgcccacatcactcaagacgtaaattatggc

SEQ ID NO: 73 - DNA, 50 bp, Homo sapiens
caaccgccttttcatcaatcgcccacatcactccagacgtaaattatggc

SEQ ID NO: 74 - DNA, 50 bp, Homo sapiens
caaccgccttttcatcaatcgcccacatcactcgtgacgtaaattatggc

SEQ ID NO: 75 - DNA, 50 bp, Homo sapiens
caaccgccttttcatcaatcgcccacatcactcgcgacgtaaattatggc

SEQ ID NO: 76 - DNA, 50 bp, Homo sapiens
caaccgccttttcatcaatcgcccacatcactcgggacgtaaattatggc

SEQ ID NO: 77 - DNA, 50 bp, Homo sapiens
caaccgccttttcatcaatcgcccacatcactcgatacgtaaattatggc

SEQ ID NO: 78 - DNA, 50 bp, Homo sapiens
caaccgccttttcatcaatcgcccacatcactcgaaacgtaaattatggc

SEQ ID NO: 79 - DNA, 50 bp, Homo sapiens
caaccgccttttcatcaatcgcccacatcactcgacacgtaaattatggc

SEQ ID NO: 80 - DNA, 50 bp, Homo sapiens
caaccgccttttcatcaatcgcccacatcactcgagccgtaaattatggc

SEQ ID NO: 81 - DNA, 50 bp, Homo sapiens; identical to SEQ ID NO: 80

SEQ ID NO: 82 - DNA, 50 bp, Homo sapiens
caaccgccttttcatcaatcgcccacatcactcgaggcgtaaattatggc

SEQ ID NO: 83 - DNA, 50 bp, Homo sapiens
caaccgccttttcatcaatcgcccacatcactcgagatgtaaattatggc

SEQ ID NO: 84 - DNA, 50 bp, Homo sapiens
caaccgccttttcatcaatcgcccacatcactcgagaagtaaattatggc

SEQ ID NO: 85 - DNA, 50 bp, Homo sapiens
caaccgccttttcatcaatcgcccacatcactcgagaggtaaattatggc

SEQ ID NO: 86 - DNA, 50 bp, Homo sapiens
caaccgccttttcatcaatcgcccacatcactcgagacttaaattatggc

SEQ ID NO: 87 - DNA, 50 bp, Homo sapiens
caaccgccttttcatcaatcgcccacatcactcgagacataaattatggc

SEQ ID NO: 88 - DNA, 50 bp, Homo sapiens
caaccgccttttcatcaatcgcccacatcactcgagacctaaattatggc

SEQ ID NO: 89 - DNA, 50 bp, Homo sapiens
caaccgccttttcatcaatcgcccacatcactcgagacgaaaattatggc

SEQ ID NO: 90 - DNA, 50 bp, Homo sapiens
caaccgccttttcatcaatcgcccacatcactcgagacgcaaattatggc

SEQ ID NO: 91 - DNA, 50 bp, Homo sapiens
caaccgccttttcatcaatcgcccacatcactcgagacggaaattatggc

SEQ ID NO: 92 - DNA, 50 bp, Homo sapiens
caaccgccttttcatcaatcgcccacatcactcgagtttaaaattatggc

SEQ ID NO: 93 - DNA, 50 bp, Homo sapiens
caaccgccttaaattcaatcgcccacatcactcgagacgtaaattatggc

SEQ ID NO: 94 - DNA, 50 bp, Homo sapiens
caaccgccttttcaaatttcgcccacatcactcgagacgtaaattatggc

SEQ ID NO: 95 - DNA, 50 bp, Homo sapiens
caaccgccttttcatcaatcgcccacatcacaaatgacgtaaattatggc

SEQ ID NO: 96 - DNA, 50 bp, Homo sapiens
caaccgccttattttaattttaattttaattttaattttaaaattatggc

(19) United States
(12) Patent Application Publication
(10) Pub. No.: US 2010/0129796 A1
(43) Pub. Date: May 27, 2010

(54) DYE PROBE FLUORESCENCE RESONANCE ENERGY TRANSFER GENOTYPING

(76) Inventors: Micah Halpern, Liberty, MO (US); Philip M. Ellis, Melbourne, FL (US)

Correspondence Address: HUSCH BLACKWELL SANDERS LLP, 4801 Main Street, Suite 1000, KANSAS CITY, MO, 64112, US

(21) Appl. No.: 12/276,849

(22) Filed: Nov. 24, 2008

(51) Int. Cl. C12Q 1/68

(52) U.S. Cl. 435/6

(57) ABSTRACT

An improved method for detecting, identifying and screening single polynucleotide polymorphisms, insertion/deletion loci, and microsatellites is provided. The method includes adding a donor intercalating dye to a sample containing an amplified target nucleic acid sequence, adding a probe containing an acceptor fluorophore to the sample, hybridizing the probe to the target sequence, exciting the donor dye with a specific wavelength of light, monitoring fluorescence from the sample due to FRET energy transfer from the dye to the probe fluorophore associated with one or both of the hybridization of the probe to the target sequence and the dissociation of the probe from the target sequence, and analyzing the sample using a melt-curve analysis to identify at least one single (or multiple) known or unknown nucleotide polymorphism, insertion/deletion loci, or microsatellite therein.

### BACKGROUND OF THE INVENTION

Discovering, screening and associating changes in DNA sequences have a significant impact across a broad range of disciplines including forensics, medicine, ecology and molecular biology. In particular, establishing differences between DNA samples from two different sources or even from the same source, under different developmental or environmental conditions, is very useful. Subtle differences in the genetic material can often yield valuable information to enable understanding of physiological processes as well as providing powerful techniques having a broad range of applications including, but not limited to, forensic science, determination of predisposition of individuals to certain diseases, tissue typing, response to pathogens, chemicals, drugs, vaccines and other agents, genetic association studies, crop and livestock breeding, ecological studies, identity testing, molecular taxonomy, and the like.

DNA technology and sequencing advances have also resulted in a significantly increased need to detect and/or quantify single nucleotide polymorphisms (SNPs), insertion/deletions (INDELs), and microsatellite variations. A SNP is a genetic marker resulting from a variation in sequence at a particular position within a DNA sequence. SNPs can result from a base transition (purine to purine or pyrimidine to pyrimidine) or transversion (purine to pyrimidine or pyrimidine to purine). INDELs typically arise when one or more nucleotides is added or subtracted from a sequence (e.g., CCT to CT). Microsatellites, also termed short tandem repeats (STR) in the forensics field, are an example of a specific type of INDEL often attributed to polymerase slippage and consist of repeating units of 1-6 base pairs (e.g., CAGCAG).

Such variation is extensive throughout all genomes. For example, the human genome consists of approximately 3 billion base pairs and inherited genetic differences contribute to human phenotypic diversity. The most common type of human genetic variation is the SNP. These single nucleotide changes are the result of normal cellular operations (a malfunction during the replication of DNA causes the wrong base to be inserted into a nucleic acid chain) or random interactions with the environment (the action of mutagens can cause chemical modification of a nucleic base (changing it into a different base). Cancers arise from the accumulation of inherited polymorphisms and/or sporadic somatic polymorphisms in the DNA that affects cell cycle and growth signaling genes. Emerging applications for which SNP, INDEL, and/or microsatellite testing is fast becoming critical include the fields of in vitro diagnostics, clinical diagnostics, molecular biology research, forensic science, identity testing, pharmacogenetics, veterinary diagnostics, agricultural-genetics testing, environmental testing, food testing, industrial process monitoring, insurance testing and others. There are many other potential applications for the detection and/or quantitation of individual/strain identification.

A wide variety of technologies have been developed to screen for these changes and fall under the major categories of hybridization-based, enzyme-based, post-amplification detection and different forms of DNA sequencing. In hybridization-screening, developments aimed at discovering and identifying DNA changes can be classified under two major sub-categories of generic DNA intercalator techniques and strand specific hybridization.

The first subcategory within hybridization includes generic methods that utilize DNA intercalating dyes that exhibit increased fluorescence when bound to double stranded DNA. These fluorescent moieties include SYBR, SYTO and a host of other well characterized dyes. End point melting curve analysis using these dyes is able to discriminate artifacts (i.e., primer dimer) from specific amplicons but maintain a somewhat low level resolution between amplicons with a similar sequence. In other words, application of dye-based hybridization methods are primarily used for PCR optimization and, only more recently, have been developed for higher resolution screening using more proprietary dyes (e.g., LC Green) and advances in data analysis. Although somewhat limited in its ability to resolve many different types of changes in DNA between samples, the major benefit to this hybridization-based approach is the cost savings associated with minimized reagent requirements and reduced design constraints.

The second subcategory within hybridization-based screening technology includes strand specific methods that utilize additional nucleic acid reaction components (beyond generic dyes) to monitor the progress of amplification reactions. The most typical added reaction component is some form of oligonucleotide probe designed in or around the sequence of interest. These methods often use fluorescence energy transfer (FET) as the basis of detection. One or more nucleic acid probes are labeled with fluorescent molecules, one of which is able to act as an energy donor and the other of which is an energy acceptor molecule. These are also referred to as a reporter molecule and a quencher molecule, respectively. The donor molecule is excited with a specific wavelength of light that falls within its excitation spectrum which causes it to emit light within its fluorescence emission wavelength. The acceptor molecule is then excited at the emitting wavelength of the first molecule by accepting energy from the donor molecule by a variety of distance-dependent energy transfer mechanisms. A specific example of FET is Fluorescence Resonance Energy Transfer (FRET). In FRET, the acceptor molecule accepts the emission energy of the donor molecule when they are in close proximity (e.g., on the same, or on a neighboring molecule) with the distance of separation termed the Forster distance. The basis of FRET detection is to monitor the changes at the acceptor emission wavelength caused by separation of the two moieties. There are two commonly used types of FRET probes: those using hydrolysis of nucleic acid probes to separate donor from acceptor, and those using hybridization to alter the spatial relationship of donor and acceptor molecules.

Hydrolysis probes are commercially available as Taqman probes generally shown in FIG. 1. These consist of DNA oligonucleotides that are labeled with donor and acceptor molecules. The probes are designed to bind to a specific region on one strand of a PCR product. Following annealing of the PCR primer to this strand, Taq enzyme extends the DNA with 5′ to 3′ polymerase activity. Taq enzyme also exhibits 5′ to 3′ exonuclease activity. TaqMan probes are typically protected at the 3′ end to prevent extension. If the TaqMan probe is hybridized to the product strand, the Taq polymerase enzyme will subsequently hydrolyze the probe thereby liberating the donor from the acceptor as the basis of detection. The signal in this instance is cumulative wherein the concentration of free donor and acceptor molecules increasing with each cycle of the amplification reaction. This approach is typically used for quantitation and more recently has been adapted for SNP detection on an assay specific basis.

As opposed to hydrolysis probes, hybridization probes are available in a number of forms and are not consumed during detection as shown in FIG. 2. Molecular beacons are an example of oligonucleotides that have complementary 5′ and 3′ sequences such that they form hairpin loops. Terminal fluorescent labels must be in close proximity for FRET to occur when the hairpin structure is formed. Following hybridization of a molecular beacon to a complementary sequence, the fluorescent labels are separated so FRET does not occur thereby forming the basis of detection. Another approach to using hybridization probes utilizes a pair of labeled oligonucleotides commonly known as dual hybridization probes. These hybridize in close proximity on a PCR product strand bringing donor and acceptor molecules together so that FRET can occur. Variations on this approach can include using a labeled amplification primer with a single adjacent probe. As opposed to dye-based hybridization, hybridization probes have shown good success with obtaining high levels of resolution for SNP genotyping but suffer from other shortcomings.

The use of either dual hybridization probes or molecular beacons requires labeling with two fluorescent molecules which subsequently increases the cost involved in using these approaches. In addition, both methods require the presence of a reasonably long stretch of a known sequence so that the probe/probe pair can bind specifically in close proximity to each other. This can be a problem in some applications wherein the length of known sequences that can be used to design an effective probe may be relatively short. Furthermore, the use of pairs of probes involves more complex experimental design whereby the genotype is a function of the melting off of both probes and requires careful design parameters often limited by sequence identity.

The most significant shortcoming to all current forms of discovering and screening changes in DNA, whether by dye or probe, is the lack of application of hybridization-based approaches for genotyping multiple (SNP, INDEL and microsatellite) types of DNA changes. Moreover, no adequate technology has been described that is amendable for both discovery and screening applications. To meet this demanding application, a technology would need to be able to identify any change within a sequence and be cost effective enough for subsequent screening of large sample numbers. The current approaches described above are good for discovery or screening but are subject to weaknesses for a combined approach due to throughput, cost, speed, and the like. In addition, current screening approaches target a single base change per assay and require prior knowledge of, for example, a particular SNP's location.

The most discriminatory markers currently used in forensic laboratory analysis are the extensively validated collection of STRs comprising the CODIS loci. The standard approach for analysis of these markers is multiplex amplification followed by capillary electrophoresis (CE) size separation. Additional methods for size discrimination including array-based hybridization and mass spectrometry have been explored, but all current approaches are subject to weaknesses in one or more of interpretation, portability, ease-of-use, cost and speed. A variety of known experimental artifacts are possible with CE-based STR genotyping including stutter peaks, non-template 3′ nucleotide addition, matrix artifacts and electronic spikes or dye ‘blobs’. Data interpretation of CE-analyzed samples can be a challenge for laboratory-trained analysts in a controlled setting. These challenges would only be expected to be exacerbated in a crime scene setting. A unique approach is required to overcome these technical and logistical hurdles.

Thus, traditional methods cannot meet the growing demand for methods that allow for rapid discovery in large sequences and complete genomes and simultaneous capabilities for screening large sample numbers for many types of DNA changes. With regard to the increasing importance of SNPs, INDELs, microsatellites and their analysis (e.g., for medical diagnosis), simple methods using relatively fast and cost effective fluorogenic techniques are highly desirable. Moreover, it would be beneficial to provide a method of SNP, INDEL and/or microsatellite identification that reduces equipment costs (e.g., capillary electrophoresis, microarrays, etc), labor times, material costs and is transferable for high throughput, point-of-care and portable applications.

### SUMMARY OF THE INVENTION

In one of many illustrative, non-limiting aspects of the present invention, there is provided a method for detecting and screening at least one SNP, at least one INDEL and/or at least one microsatellite. The method includes asymmetrically amplifying a target nucleic acid in the presence of a DNA intercalating dye, adding a probe containing an acceptor fluorophore to the sample, hybridizing the probe to the target sequence, and analyzing the sample using a melt-curve analysis to identify at least one SNP, at least one INDEL, and/or at least one microsatellite therein. For ease of understanding, the methods of the present invention will be primarily described in reference to detection and screening of SNPs. However, it will be appreciated by those skilled in the art that the methods hereof may also be used in connection with INDELs and microsatellites or the like without departing from the scope of the present invention or requiring undue experimentation.

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers may be designed to hybridize to either strand and SNP genotyping methods disclosed herein may generally target either strand.

For purposes of the invention, the term “complementary” means having the ability to hybridize to a genomic region, a gene, cDNA, or an RNA transcript thereof as the result of base-specific hydrogen bonding between complementary strands to form Watson-Crick or Hoogstein base pairs. As used herein, “hybridize” refers to the formation of a base-paired interaction between single-stranded nucleic acid molecules.

According to the invention, where complementary sequences hybridize to one another then the hybridization conditions are such that two nucleotide sequences with an exact match for base pairing, or only a small percentage (1-40%) of base mismatch between the two sequences, form a base paired double-stranded nucleic acid molecule that is stable enough to allow detection. Thus, according to the invention, for two single-stranded nucleic acid molecules to hybridize to one another to form a double-stranded nucleic acid molecule, their nucleotide sequences form at least 60% base pairing of the nucleotides of the shorter of the two single-stranded nucleic acid molecules, or at least 95% base pairing, or at least 98% base pairing, or at least 99% base pairing, or form 100% base pairing of the nucleotides of the shorter of the two single-stranded nucleic acid molecules.

The terms “polynucleotide” and “oligonucleotide” mean polymers of nucleotide monomers, including analogs of such polymers, including double- and single-stranded deoxyribonucleotides, ribonucleotides, α-anomeric forms thereof, and the like. Polynucleotides and oligonucleotides can be of any length.

“Primers” are oligonucleotides that comprise sequences that are employed in a reaction to facilitate polymerization of the primer and at least one additional nucleotide. Polymerization may be carried out for purposes of amplification, primer extension, and/or sequencing. Primers may be oligonucleotides that are designed to hybridize with a portion of the target nucleic acid sequence or amplification products in a sequence-specific manner, and serve as primers for primer extension, amplification and/or sequencing reactions. The criteria for designing sequence-specific primers are well known to persons of skill in the art. The sequence-specific portions of the primers are of sufficient length to permit specific annealing to complementary sequences in ligation products and amplification products, as appropriate.

### BRIEF DESCRIPTION OF THE SEVERAL VIEWS OF THE DRAWING

In the accompanying drawings that form a part of the specification and that are to be read in conjunction therewith:

FIG. 1 is a schematic representation of a TaqMan hydrolysis probe;

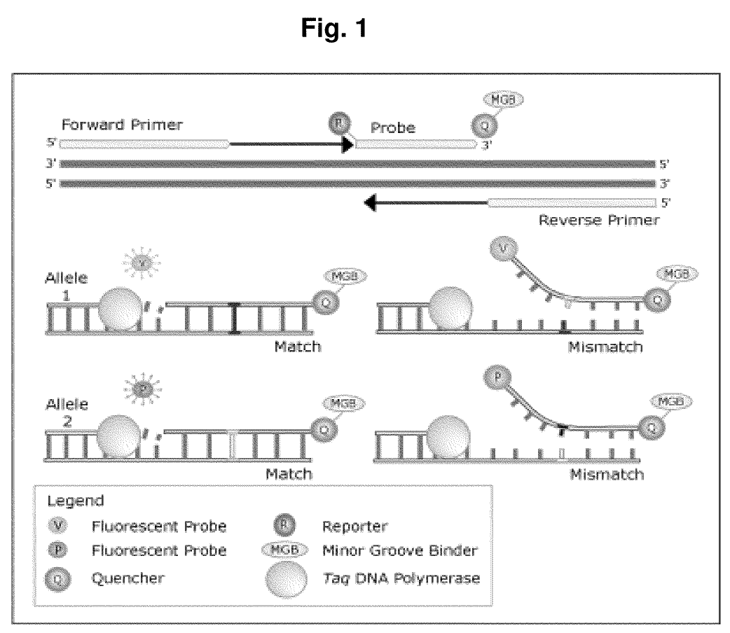

FIG. 2 is a schematic representation of hybridization probes;

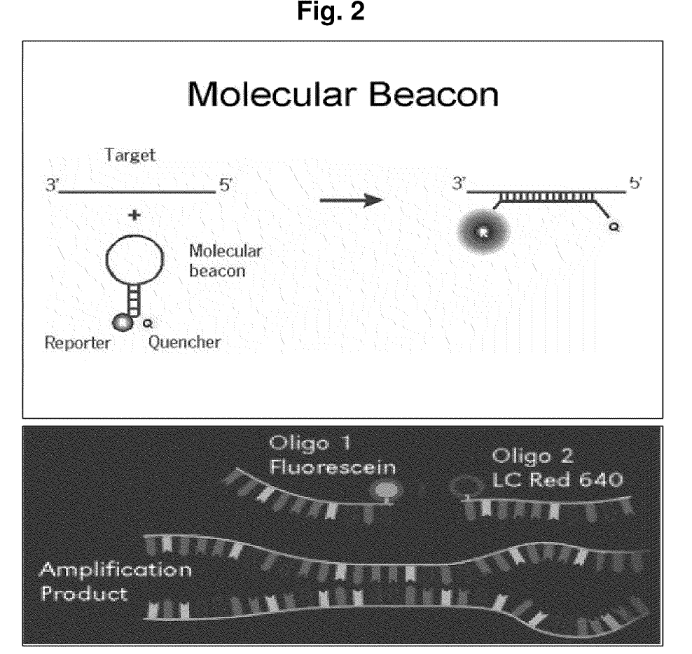

FIG. 3 is a schematic representation of one embodiment of the method of the present invention;

FIG. 4 is a schematic representation of one embodiment of the method of the present invention;

FIG. 5 is a graphical representation of one example of FRET excitation and emission;

FIG. 6a is a schematic representation of a single locus probe hybridization scenario in accordance with one embodiment of the method of the present invention;

FIG. 6b is a schematic representation of a complex locus probe hybridization scenario in accordance with one embodiment of the method of the present invention;

FIG. 7 is a graphical representation of resulting melt peaks from the probe hybridzation scenarios of FIGS. 6a and 6b;

FIG. 8 is a graphical representation of data generated from synthetic resolution testing of a 30 bp probe wherein error bars at ±0.4 degrees accounts for potential differences between replicates due to thermal block temperature control;

FIG. 9 is a graphical representation of data generated from synthetic resolution testing using a 21 bp fluorophore labeled probe (top panel) and an unlabeled probe (bottom panel);

FIG. 10 is a graphical representation of data generated from synthetic resolution testing using a 15 bp fluorophore labeled probe;

FIG. 11 is a graphical representation of data generated from synthetic resolution testing of species multi-SNP templates wherein error bars at ±1.0 degrees accounts for potential differences between replicates due to thermal block temperature control;

FIG. 12 is a graphical representation of data generated from synthetic resolution testing of a probe labeled by tDt wherein error bars at ±0.4 degrees accounts for potential differences between replicates due to thermal block temperature control;

FIG. 13 is a graphical representation of data generated from probe treatments of a 30 bp fluorophore labeled probe with inosines including an unmodified (blue), inosine at probe position 30 (pink) and inosines at probe positions 28, 29 and 30 (green);

FIG. 14 is a graphical representation of data generated from cytochrome B speciation using one embodiment of the method of the present invention;

FIG. 15 is a graphical representation of data generated from MHCdrBeta penguin paternity testing using one embodiment of the method of the present invention;

FIG. 16 is a graphical representation of dpFRET sensitivity using quantitated human genomic standard in accordance with one embodiment of the method of the present invention;

FIG. 17 is a graphical representation of dpFRET microsatellite testing of JCCL samples for TPOX locus in accordance with one embodiment of the method of the present invention;

FIG. 18 is a graphical representation of dpFRET microsatellite testing (D3 complex locus) in accordance with one embodiment of the method of the present invention;

FIG. 19 is a graphical representation of dpFRET microsatellite testing (lack of allelic dropout) in accordance with one embodiment of the method of the present invention;

FIG. 20 is a graphical representation of dpFRET microsatellite testing (mixed samples) in accordance with one embodiment of the method of the present invention;

FIG. 21 is a graphical representation of dpFRET microsatellite testing (chimeric bone marrow transplant sample) in accordance with one embodiment of the method of the present invention;

FIG. 22 is a schematic representation of relative sequencing in accordance with one embodiment of the method of the present invention;

FIG. 23 is a represenation of a gel-electrophoresis showing dpFRET 80 cycle CytB amplification wherein specific product (350 bp) and non-specific product are shown;

FIG. 24 is a graphical representation of a 9 repeat probe mismatch peak differentiation in accordance with one embodiment of the method of the present invention;

FIG. 25 is a graphical representation of a 11 repeat probe mismatch peak differentiation wherein yellow hatched lines delineate approximate melt temperatures for different alleles using a single repeat probe

FIG. 26 is a chi-square curve fitting a 9 repeat probe in accordance with one embodiment of the method of the present invention;

FIG. 27 is a chi-square curve fitting an 11 repeat probe in accordance with one embodiment of the method of the present invention;

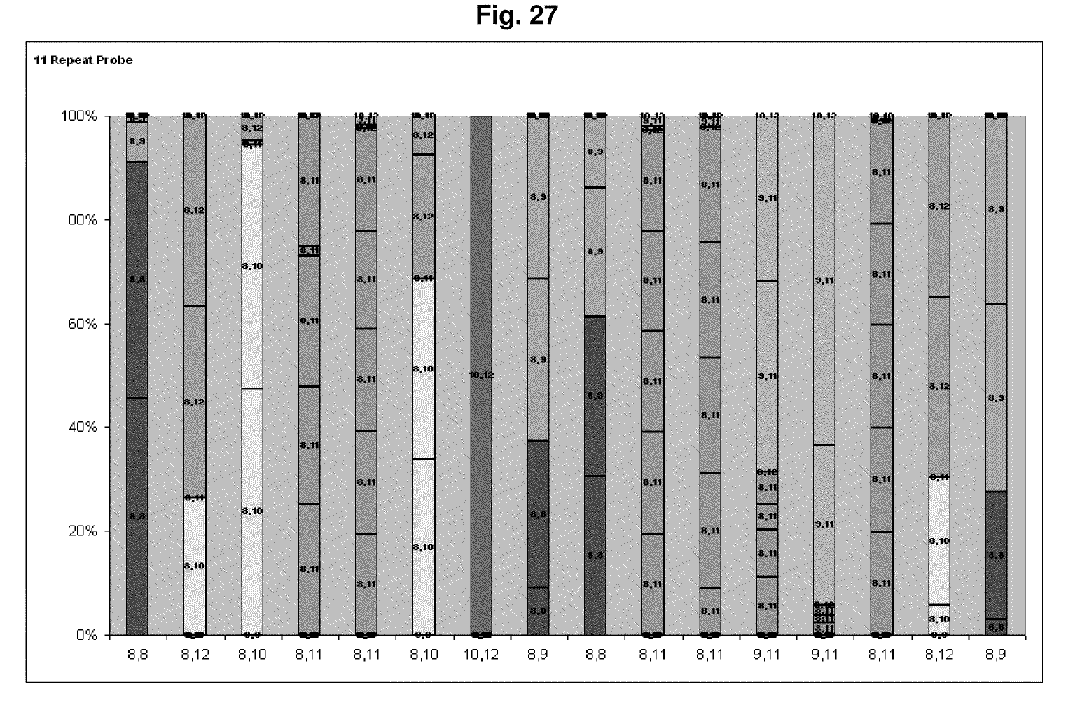

FIG. 28 is a graphical representation of a slope ratio analysis in accordance with one embodiment of the method of the present invention; and

FIG. 29 is a graphical representation of the loss of heterozygosity in cancer in accordance with one embodiment of the method of the present invention.

### DETAILED DESCRIPTION OF THE INVENTION

There is provided herein a method for the identification and classification of detecting and screening at least one single nucleotide polymorphism (SNP), at least one insertion/deletion loci (INDEL), and/or at least one microsatellite using an asymmetric polymerase chain reaction (PCR) and a fluorescence resonance energy-transfer (FRET) between an intercalating dye and a fluorophore labeled probe. The method of the present invention is used for detecting and identifying at least one SNP, INDEL, or microsatellite having a known or, in particular, an unknown location in a DNA sequence. The present method may be used on any source of DNA or cDNA prepared from RNA derived from a source including, but not limited to, fungal, plant, yeast, bacterial, viral, human, animal, any living organism or combinations thereof (collectively hereinafter “DNA source”). The methods of the present invention may be used on both dead and live cultured organisms. It will be appreciated by one skilled in the art that the methods of the present invention may be used to analyze any source of DNA by adjusting variables described hereinbelow including primers and probes. Moreover, it is contemplated that the present invention may be applied to virtually any nucleic acid. The methods hereof are particularly useful for detecting and screening single or multiple nucleotide substitutions in a target.

In accordance with certain embodiments of the methods of the present invention, a clean sample of DNA is extracted from the desired DNA source. Extraction techniques are well known to those skilled in the art and any such technique may be used to extract the desired DNA from a DNA source for analysis. As an illustrative example, an aliquot of DNA, preferably in a range of about 0.001 nanograms to about 10000 nanograms, more preferably from about 0.1 to 1000 nanograms, and most preferably from about 1 to 100 nanograms is extracted from a DNA source. In order to analyze the DNA sample, however, the chromosomal sequences and, in particular, the target nucleic acid sequence must first be amplified.

Nucleic acid amplification strategies have been widely developed because of their ability to amplify the number of copies of the chromosomal sequences to be analyzed. Different in vitro nucleic acid amplification systems are known in the art. Among them, the polymerase chain reaction (PCR) method is the most popular because PCR is a quick and relatively easy method for generating unlimited copies of any fragment of DNA. It exploits the natural function of the enzymes known as polymerases. These enzymes are present in all living organisms and copy the genetic material and also proofread and correct the copies. PCR requires a template molecule (the DNA or RNA) to be amplified and two primers. PCR involves three basic steps. First, the target nucleic acid sequence is denatured. That is, the strands of its helix is unwound and separated by heating the sequence to a temperature of from about 90-100° C. The second step is hybridization or annealing, in which the primers bind to their complementary bases on the single-stranded nucleic acid sequence. The third step is nucleic acid synthesis by a polymerase. Starting from the primer, the polymerase can read a template nucleic acid strand and match it with complementary nucleotides present in the reaction mixture. The results is two new helixes in place of the first, each composed of one of the original strands plus its newly assembled complementary strand. After denaturation, this process may be repeated over and over again, leading to an exponential increase of the target nucleic acid sequence.

In certain embodiments of the methods of the present invention, asymmetric PCR is used to amplify one strand of DNA from an original DNA sample more than the other strand. In an illustrative example, from about 10 femtograms to 10 micrograms of original DNA sample may be used. In asymmetric PCR, PCR is carried out as usual but with a great excess of the first primer complementary for the strand desired to be amplified (i.e., containing the target nucleic acid sequence). This occurs by adding a disproportionately higher amount of the first amplification primer than the second amplification primer. In an illustrative example, the primer concentration ratio may be from about 1:5. However, one skilled in the art will appreciate that any suitable primer concentration ratio may be utilized without departing from the scope of the present invention. The presence of the excess first primer results in the DNA strand containing the target nucleic acid sequence continuing to amplify after its other (non-target) complementary DNA strand has run out of the second primer and no longer replicates. The strand that is generated is determined by which of the first or second amplification primer is added in higher amounts. In this manner, many copies of a single-stranded target nucleotide sequence may be generated.

There are two amplification phases that can be combined in different ways to achieve asymmetric PCR amplification. The first phase employs two primers involved in a standard PCR amplification to increase the concentration of a double stranded copy of the region of interest. The second phase employs a single primer (that can be the same or different from the primers employed in the first phase) complementary to the strand termed the hybridization template strand. Different combinations of these two phases of amplification are possible. One embodiment is represented by combining the two phases in a single reaction tube as typically described for asymmetric PCR. This approach is useful when there is an abundance of sample and only requires testing with a single hybridization probe as in the case of screening for a single known polymorphism. Another embodiment consists of phase I double stranded amplification (either in singleplex or multiplex fashion), aliquoting of phase I products into single or multiple phase II single stranded amplification reactions. This approach is useful for limited amounts of sample or genotyping across large sequence distances. This reduces the amount of sample and reagents required to produce the initial double stranded template.

Methods of optimizing amplification reactions are well known to those skilled in the art. For example, it is well known that PCR may be optimized by altering times and temperatures for annealing, polymerization, and denaturing, as well as changing the buffers, salts, and other reagents in the reaction composition. Optimization can also be affected by the design of the amplification primers used. For example, the length of the primers, as well as the G-C:A-T ratio can alter the efficiency of primer annealing, thus altering the amplification reaction. It will be appreciated that other methods now known or hereafter discovered for isolating and amplifying a DNA strand may be used instead of asymmetric PCR to produce the hybridization template strand.

Following amplification and generation of a hybridization template, a fluorophore labeled oligonucleotide probe is hybridized to the template in the presence of a DNA intercalating dye. The exact fraction of the nucleotides that must be complementary in order to obtain stable hybridization varies with a number of hybridization condition factors, including, without limitation, nucleotide sequence (e.g., G and C content of the shorter of the two single stranded nucleic acid molecules), the location of the mismatches along the two molecules, salt concentrations of the hybridization buffers, temperature, and pH. Formulae for estimating the melting temperature (Tₘ) of a double-stranded nucleic acid molecule (i.e., the temperature at which the double-stranded molecule becomes single stranded) are well known by those skilled in the art. The probe can either be commercially synthesized or chemically/enzymatically created in the lab using a number of known labeling techniques including terminal transferase and other labeling strategies.

In a first scenario for probe hybridization, Phase I (double stranded), Phase II (single stranded amplification) and probe hybridization are accomplished in the same closed tube reaction. This is done by physically blocking the 3′ end of the probe (fluorophore, phosphate, ddNTP, etc.) to prevent polymerase extension of the probe during amplification. This approach is useful for rapid genotyping of the sequence complementary to a single or small number (generally less than five) of probes in a single reaction for a limited number of targets. The second scenario consists of Phase I and Phase II amplification in a single reaction followed by hybridization with a single or small number of probes. This approach is similar to the first scenario but alleviates the added step of blocking the 3′ end of the probe and permits fluorophore labeling of the 5′ end. Residual amplification by polymerase extension in the follow-on hybridization reaction is subsequently blocked by addition of substances (EDTA) inhibitory to PCR that do not interfere with DNA hybridization. The third scenario consists of phase I amplification in a singleplex or multiplex fashion to amplify a single or multiple targets. This reaction is then aliquoted across a single or multiple phase II amplification reaction that is either supplemented with 3′ blocked probes or supplemented post-reaction with probes and a PCR inhibitor.

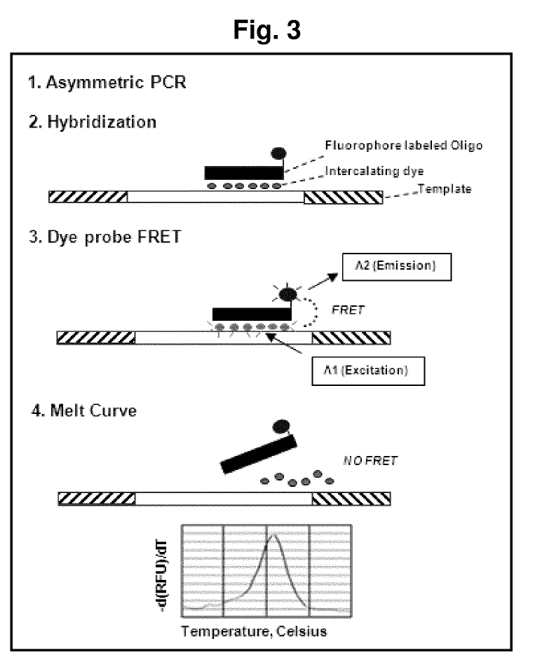

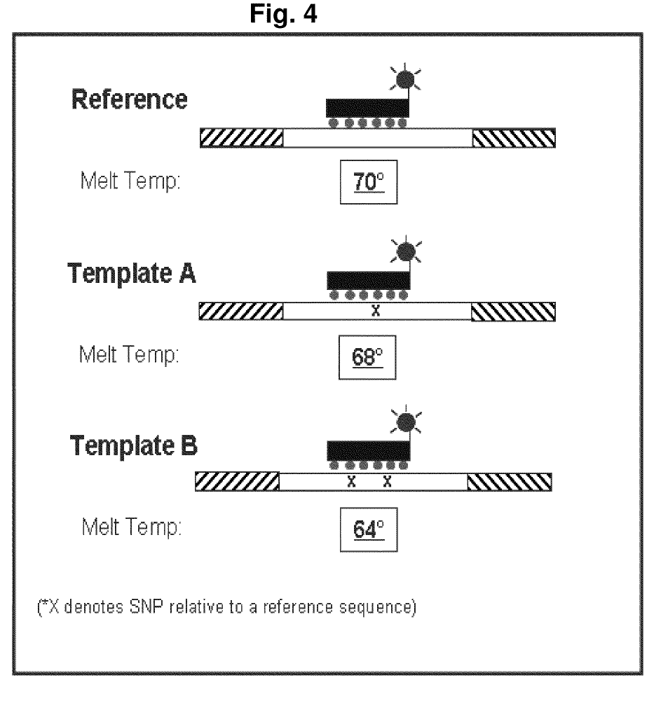

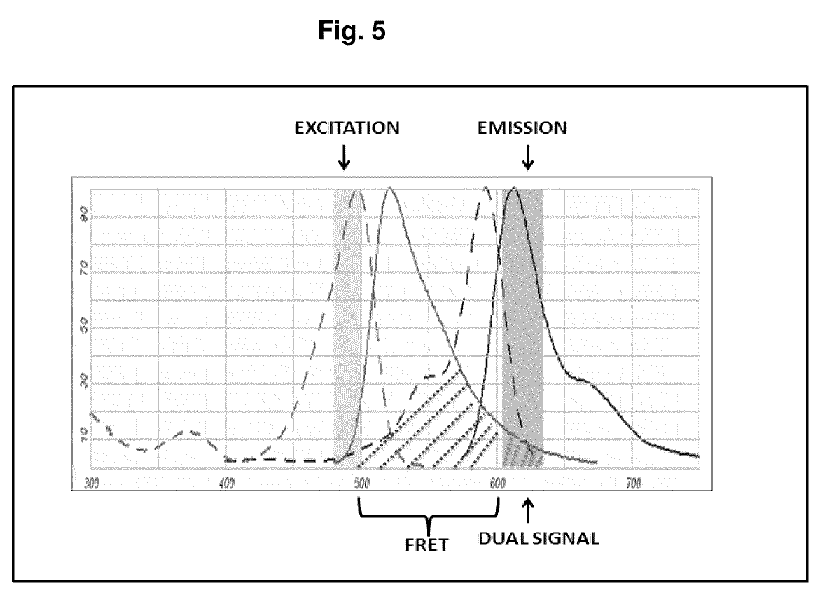

Following probe hybridization, the next step involves detecting the sample's DNA hybridization status using fluorescence resonance energy transfer (FRET). FRET is particularly useful because it enables specification of a target sequence and has the potential for multiplexing. The basic goal of this procedure is to produce signals indicative of the presence of a single SNP in the double-stranded DNA as shown in FIG. 3 or multiple SNPs as shown in FIG. 4 wherein these signals change or disappear when the DNA becomes single-stranded following denaturation. In FRET, a donor fluorophore molecule absorbs excitation energy and delivers this via dipole-dipole interaction to a nearby acceptor fluorophore molecule. The acceptor fluorophore then reemits the energy at a higher wavelength. This process only occurs when the donor and acceptor molecules are sufficiently close to one another. Several different strategies for determining the optimal physical arrangement of the donor and acceptor moieties are known in the art, any of which may be used in the present invention. Thus, if the donor and acceptor molecules are in proximity to one another, the acceptor molecule reemits the fluorescent signal of the donor molecule following excitation. However, when the two molecules are held apart from one another, only the fluorescence of the donor molecule can be detected with no fluorescence detected from the acceptor fluorophore. It will be appreciated by those skilled in the art that many different dye/fluorophore combinations are possible and suitable for use in the present invention. For example, any suitable donor fluorophore, such as SYBR Green I (Excitation 490, Emission 520), and any suitable acceptor fluorophore, such as Texas Red (Excitation 590, Emission 620), may be used in the present invention. So, for example, the donor fluorophore may be excited at a wavelength of 490 nm, emits at a wavelength of 520 nm which is then transferred via FRET to the acceptor fluorophore on the labeled probe and reemitted at 620 nm. FIG. 5 shows excitation and emission wavelengths for SYBR Green I, and Texas Red, the region of FRET between the two molecules, and the dual emiussion signal generated by both dyes.

In certain embodiments, the next step of the methods of the present invention involves the detection of single or multiple SNPs within the target nucleotide with follow-on differentiation by melt-curve variation between different sequences. It will be appreciated by those skilled in the art that one of the beneficial outcomes of this approach is the generation of two melt peaks. As discussed herein, the peak (or multiple peaks as is the case for a heterozygote) at the lower melt temperature is the result from the FRET probe and the peak at a higher temperature is the result from the melting of the amplicon itself. The amplicon melt peak is generated by fluorescence of intercalated SYBR Green I at the tail end of the SYBR Green I emission spectrum. This secondary melt peak provides a positive signal for amplification of specific product and can be used to distinguish non-specific signal occasionally generated by the probe for >45 cycle amplification reactions.

This approach has been tested for its ability to detect and differentiate between different and multiple SNPs within a target region using a single labeled probe as shown in FIGS. 3 and 4. Due to melting behaviors of DNA, positioning of the signal generating molecule at the end of the amplicon (rather than throughout the strand as in the case of intercalating dyes) capitalizes on the “end effects” seen for DNA melting. Intercalating dyes bind across the amplicon and signal differences due to SNPs at different positions can be reduced due to a signal blending effect. Minor differences in melting are amplified across the strand as it melts when end effects are monitored using the dpFRET approach.

In particular, one embodiment of the method of the present invention provides that both strands of the hybridized combined DNA probe/template hybrid discussed hereinabove are denatured at a high temperature. The temperature is then lowered to a point where of the template strand and fluorescent labeled probe reassemble and the donor intercalating dye is deposited between the template and the probe. The reaction is exposed to a specific wavelength of light and as a result of intercalation, the dye fluoresces at a particular wavelength. This energy is transferred by FRET to the fluorophore on the hybridized probe, absorbed and reemitted at a different wavelength than the intercalating dye. Readings are taken as the temperature is slowly increased in order to detect single or multiple base pair changes within the target nucleotide. As the temperature reaches a critical point where the two strands of the fragment begin to break apart, the donor fluorophore is released into solution where it no longer fluoresces. This decreases the signal of the hybridization probe acceptor fluorophore and a melt point is achieved when 50% of the product denatures.

Melt point temperatures are dependent on the template sequence. A perfect match between the probe sequence and template sequence produces a distinctive melt temperature. Single and multiple (1-40%) SNPs in the sequence of the template result in a reduced melt temperature in comparison to a perfect match. This means that, using a single probe sequence, one or multiple changes within a portion of template sequence can be detected as shown in FIGS. 3 and 4. Subsequent melt temperatures depend on what and how many changes occur within a sequence. In essence, one obtains a sequence genotype relative to a reference sequence. The lower the temperature the more SNPs are present. This permits detection of any change at any position in the template relative to the probe sequence. By interrogating successive probes (in separate or the same (multiplex dyes/fluorophores) hybridization reactions) along a sequence, any size region of sequence for single or multiple SNPs can be scanned. This describes the SNP discovery aspect of certain embodiments of the present invention and has been termed relative sequencing. Once a SNP under a particular probe is discovered, that same probe can then be used to test multiple samples for high throughput screening. This describes the SNP screening aspect of the approach.

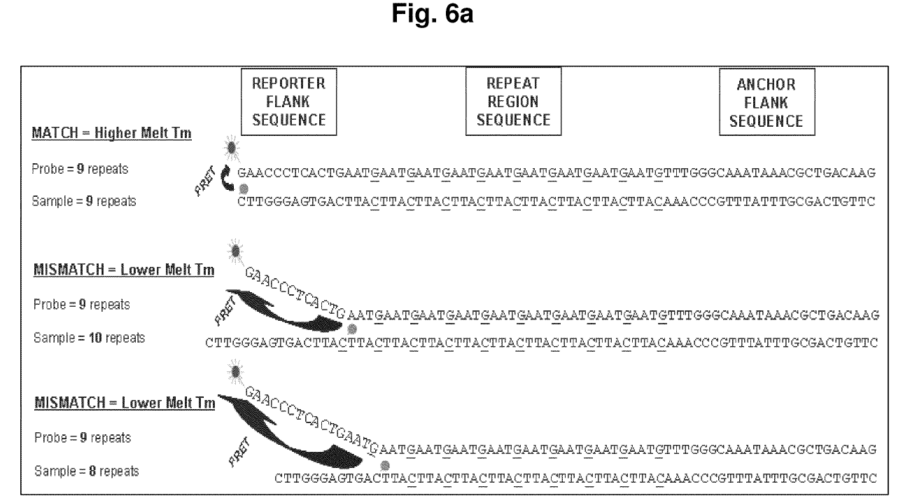

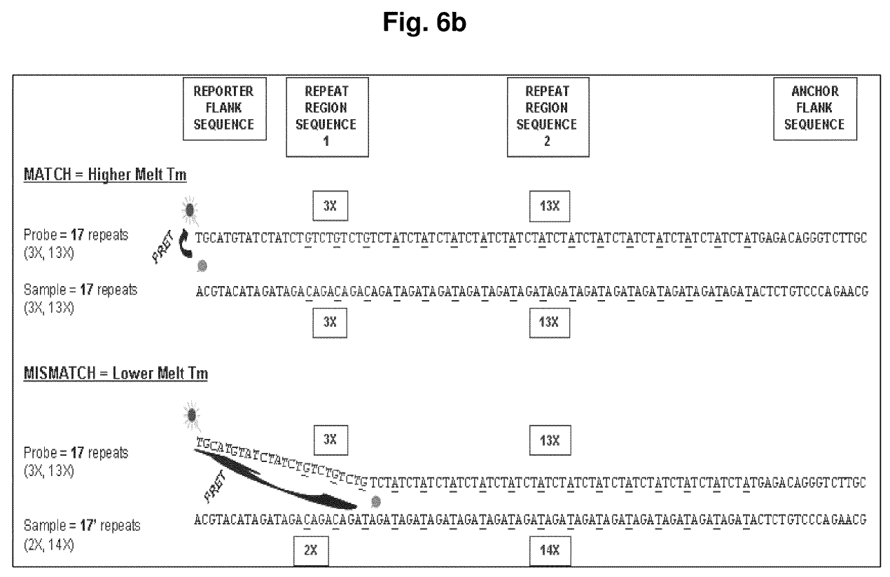

It will be appreciated by those skilled in the art that the melt temperature of the probe/template hybrid can be artificially manipulated using a number of approaches including additives (DMSO, etc.) and, more importantly, using nucleotide analogues incorporated into the probe. An example is provided in FIG. 6 wherein inosine bases are incorporated into the probe at different positions to alter the melt behavior of probe against different template sequences. This would allow manipulation of probe sequence composition in a way that could produce a unique melt signature for any and all changes within the template sequence.

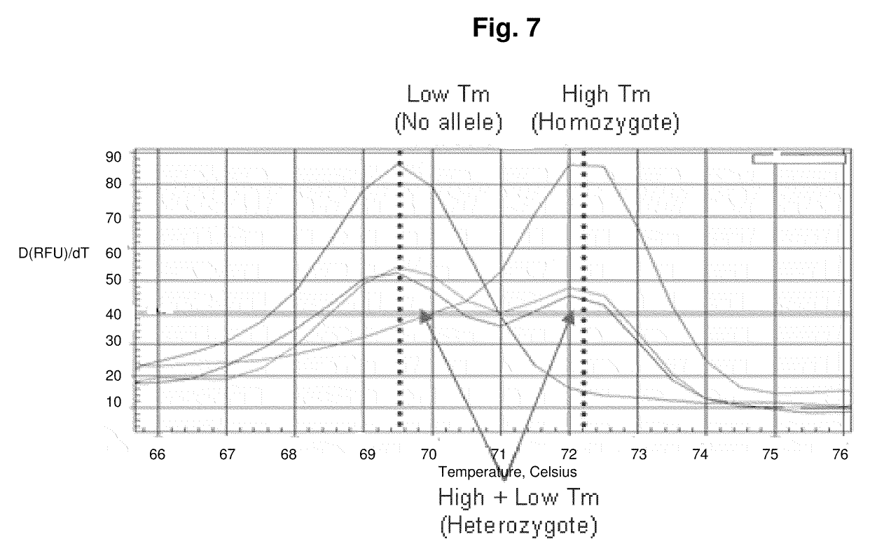

A significant strength of the inventive dpFRET methods of the present invention is that the same technology used for SNP detection and screening can be applied to the typing of microsatellites or repetitive sequences and insertion/deletion loci. In a similar manner as SNP detection, a template is generated for a region of interest by asymmetric PCR followed by hybridization with an allele specific probe. The allele specific probe contains a defined number of repeats. This approach produces two potential melt peaks for the probe consisting of either a match (High Tₘ) or a mismatch (Low Tₘ) with the number of repeats contained within the target. Similar to the SNP methods discussed hereinabove, a second melt peak at a higher temperature signifies production of specific amplicon. Potential probe hybridization scenarios are shown in FIG. 6a for a simple locus and FIG. 6b for a complex locus. An example of the resulting melt peaks are shown in FIG. 7.

This approach provides significant benefits over standard size separation based analysis. No additional manipulation beyond a standard melt curve is required thereby significantly reducing the time to results. The only additional costs are labeled probes which costs significantly less than the reagents required for fragment analysis. Moreover, less sample manipulation is required and the protocol is highly amendable to microfluidic and automated platforms. Most importantly, the objective analysis can be automated and does not suffer from the same potential artifacts as CE analysis.

The following examples are offered by way of illustration and not by way of limitation. It will be appreciated by those of ordinary skill in the art that any of the apparatus used herein may be substituted with other apparatus suitable for use in the methods of the present invention.

### EXAMPLES

### Example 1

### Synthetic SNP Testing

Sequences corresponding to positions 14925-14974 of the Cambridge human mitochondrial genome (J01415) and mutated templates were synthesized, purified by standard desalting, and concentrations were standardized by a commercial source (Integrated DNA Technologies). The mutated templates included representatives for substantially every possible single point mutation within the 30 bp central core region composed of positions 14935-14964. The variable animal species template library contained sequences corresponding to the same position of the Cambridge human mitochondrial genome from a number of animal species as listed in Table 1 and was generated by the same commercial source. Non-variable 10 bp sequences flanking the variable regions were also included in each template to avoid potential problems associated with incomplete synthesis such as N-1 templates. All sequences for both the human and species variation libraries are listed in appendix A.

TABLE 1
Species included in the species variation library

Human
Aardvark
AfElephant
Alpaca
Armadillo
AsBlkBear
AsElephant
AtWalrus
AuSeaLion
Baboon
BalWhale
Bat
BrnBear
Buffalo
CaspSeal
Cat
Catfish
Cattle
Cheeta
Chicken
Chimp
Coelacanth
Colobus
Coyote
Deer
Desman
Dog
Dogfish
Donkey
Dugong
Eel
Finch
FinWhale
FlyFox
Fox
Frog
GdFurSeal
MtFurSeal
Goat
Goby
Gorilla
GrayWolf
Grebe
GrnLizard
GrnMonkey
GuinPig
Hamster
Hedgehog
Heron
Hippo
Horse
HumWhale
Hyrax
Junglefowl
Kestrel
Kiwi
Langur
Lemur
Leopard
LfMonkey
Loach
Loon
LprdSeal
Mammoth
Minnow
MnkSeal
Mongoose
Mouse
Muntjac
NileCroc
Orangutan
Penguin
Pig
PolarBear
Porpoise
Rabbit
Rat
Reindeer
RghtWhale
Rhea
Rhino
RvrDolphin
Salamander
Salmon
Sheep
Skate
Sloth
SptSeal
Squirrel
Stingray
Sturgeon
TftDeer
TwnVole
Vole
WhtShark
Yak

Both template libraries were evaluated by standard melt curve analysis with human reference probe sequences (30 bp: ACGTCTCGAGTGATGTGGGCGATTGATGAA, 21 bp: TCGAGTGATGTGGGCGATTGA, 15 bp: GTGGGCGATTGATGA) and labeled at the 3′ terminus with a Texas Red-X NHS Ester. The fluorescent probe was commercially synthesized, HPLC purified and quantity standardized by a commercial source (Integrated DNA Technologies). Hybridization reactions contained 1× SYBR Green I Master Mix (Bio-Rad), 50 uM template and a 5 uM labeled probe and were subjected to the following thermal protocol on an IQ5 real-time thermal cycler (Bio-Rad): 95° C. for 1 minute, 25° C. for 1 minute and incremental increase of 0.2° C. to a final temperature of 95° C. A standard excitation filter of 490 nm (30 nm bandwidth) was coupled with a 620 nm (20 nm bandwidth) emission filter placed in the appropriate corresponding position of the emission filter wheel.

### Example 2

### Terminal Deoxynucleotidyl Transferase (TdT) Probe Labeling

Labeling of synthetic oligonucleotides was tested using TdT (New England Biolabs) and ChromaTide Texas Red-12-dUTP (Invitrogen). The same oligonucleotide sequence used for synthetic probe testing (ACGTCTCGAGTGATGTGGGCGATTGATGAA) was synthesized (Integrated DNA Technologies) followed by standard desalt purification. The following were combined for TdT labeling: 200 uM synthetic oligonucleotide, 1× NEB buffer 4, CoCl2 (5 mM), Texas Red-12-dUTP (1 mM) and 60 units of terminal transferase. The reaction was incubated overnight at 37° C. and terminated by incubation at 70° C. for 10 minutes. The reaction was subjected to DyeEX chromatographic separation of unincorporated fluorophore nucleotides (Qiagen). Probes were hybridized to the human variation template library and melted as previously described.

### Example 3

### Inosine Probes

Artificial manipulation of hybridization melt temperatures was examined through incorporation of the nucleotide analogue inosine at variable places within the same probe sequence. Two hybridization probes were synthesized with inosine at position 30 and a second probe at positions 28, 29 and 30 and fluorescently labeled with tDt as previously described to examine effects on melt behavior. Probes were hybridized to the human variation template library and melted as previously described.

### Example 4

### Assay Design, Amplification and Probe Hybridization

Cytochrome B—Species Identification

Published sequences (NCBI) encompassing Cytochrome B for multiple animal species were aligned using MegAlign (Lasergene) and regions of conservation were used to manually design primers according to standard practice. Optimal primer sequences used for dpFRET testing were CYTB 0088F Mix: 5′-TCCGCATGATGAAAyTTyGGnTC-3′ and CYTB 0438R Mix: 5′ -GTGGCCCCTCAGAAdGAyATyTG-3′. Genomic material for multiple animal species was provided by Brookfield Zoo (Brookfield, Ill.) and human and ferret genomic material was provided by the National Center for Forensic Science (Orlando, Fla.). Asymmetric PCR reactions were supplemented with 1× SYBR Green Mastermix (BioRad), 500 nM forward primer and 15 nM reverse primer. Cycling parameters consisted of the following protocol: Initial denaturation at 95° C. for 3 minutes followed by 40 cycles of 95° C. for 10 sec, 59° C. for 40 sec which formed the double stranded amplification portion of the protocol. This was immediately followed by 40 cycles of 95° C. for 10 sec, 56° C. for 40 sec forming the single stranded amplification portion of the protocol. Post amplification, 5 uM of probe complementary to human Cytochrome B sequence was added to each reaction and melted as previously described using a 0.5 degree incremental increase in temperature.

MHCdrB—Individual Identification

Published sequences (NCBI) of Mhc DRB for multiple animal species were aligned using MegAlign (Lasergene) and regions of conservation were used to manually design primers according to standard practice. Optimal primer sequences for dpFRET testing were UNIV_MHCdr_{—}3F Mix: 5′-ACGGsACsGAGCGGGTG-3′ and UNIV_MHCdr_{—}3R: 5′-CACCCCGTAGTTGTGTC-3′. Previously extracted and quantitated genomic samples derived from blood for two families of captive Humboldt Penguins (Spheniscus humboldti) were provided by Brookfield Zoo (Brookfield, Ill.). Quantitation was verified as previously described. Asymmetric PCR reactions containing 1× SYBR Green Mastermix (BioRad), 100 nM forward primer and 500 nM reverse primer were amplified using the following thermal protocol: Initial denaturation at 95° C. for 3 minutes followed by 40 cycles of 95° C. for 10 sec, 63° C. for 40 sec. This was immediately followed by 40 cycles of 95° C. for 10 sec, 59° C. for 40 sec. Following amplification, the reaction was supplemented with 5 uM of commercially synthesized Texas Red fluorescently labeled probe: UNIVdr 0245 (ATAACCAAGAGGAGTCCGTGCGCTTCGACAGCGA/3′TR), UNIVdr 0273 (5′TR/AGCGACGTGGGGGAGTACCGGGCGGTGACGGAGCTGGG), UNIVdr 0309-3′TR (GGGCGGCCTGATGCCGAGTACTGGAACAGCCAGAAGGA/3′ TR), UNIVdr 0340-3′TR (CAGAAGGACCTCCTGGAGCAGAGGCGGGCCGCGGTGGA/3′ TR), HUMdr 0509-3′TR (GGCTGAGGTGGACACGTACTGCCGA/3′ TR) and HUMdr 0536-3′TR (CACAACTACGGGGTGGTGACCCCTTTCACT/3′TR). Reactions were subjected to melt curve analysis using a 0.5 degree incremental increase in temperature on an IQ5 real-time PCR platform (Bio-Rad). Amplicons generated for dpFRET testing were also sequenced using standard dideoxy sequencing according to manufacturers protocols (Applied Biosystems) for comparison to dpFRET results.

### Example 5

### Microsatellite Assay Design, Amplification and Probe Hybridization

Human TPOX and D3S1358 primer sequences from the PowerPlex 16 kit (Promega) were commercially synthesized (Integrated DNA Technologies) and tested against CE genotyped samples derived from buccal swabs provided by the Johnson County Crime Laboratory (Olathe, Kans.). Primer sequences included: TPOX F (5′-GCACAGAACAGGCACTTAGG-3′), TPOX R (5′-CGCTCAAACGTGAGGTTG-3′), D3S1358 F (ATGAAATCAACAGAGGCTTGC) and D3S1358 R (ACTGCAGTCCAATCTGGGT). The thermal protocol used for PCR amplification consisted of the following: Initial denaturation at 95° C. for 3 minutes followed by 40 cycles of 95° C. for 10 sec, 59° C. for 30 sec and 72° C. for 30 sec followed by 40 cycles of 95° C. for 0 sec, 57° C. for 30 sec and 72° C. for 30 sec. Following amplification, each reaction was supplemented with 5 uM of commercially synthesized allele specific Texas Red labeled probe and melted as previously described using a 0.5 degree incremental increase in temperature on an IQ5 real-time PCR platform (Bio-Rad). Probes consisted of the following basic structure wherein number of core repeats (N) corresponded with each allele tested:

TPOX
[GAACCCTCACTG (AATG)N TTTGGGCAAATAAACGCTGACAAG]
D3S1358
[TGCATGTATCTA (TCTG)N (TCTA) N TGAGACAGGGTCTTGC]

### Example 6

### Sensitivity and Allelic Dropout

Human genomic samples used for STR individual identification testing were also used to determine assay sensitivity and potential for allelic dropout. Both homozygote and heterozygote samples were tested using protocols previously described for Mhc DRB and TPOX. Samples were re-quantitated using Picogreen and manufacturers protocols (Invitrogen) and diluted ten fold from 5 nanograms (approximately equivalent to 1000 genomic copies) to 500 femtograms (approximately equivalent to 0.1 genomic copies) in water using ten fold dilutions. Amplification and melt curve analysis was performed as described previously.

### Example 7

### Mixed Sample Testing

Laboratory generated mixes of human genomic samples were used to determine the potential to detect multiple STR genotypes within a mixed sample. Following quantification of material obtained from the Johnson County Crime Laboratory (described previously), 1 nanogram samples from a homozygote, heterozygote and an individual lacking a TPOX eight repeat allele were mixed in different combinations to examine the ability to detect changes in allelic concentrations within a sample.

Following laboratory generated mix testing, samples provided by the Dartmouth School of Medicine were tested for application to “Real World” samples. Samples were originally obtained for a previous study on chimerism in bone marrow transplant patients. Multiple cell fractions (donor, recipient, monocytes, granulocytes, peripheral blood and bone marrow) were sampled following treatment to monitor the success or rejection of the transplanted tissue. If transplant recipient genotype is detected in any of the cell fractions this dictates the need for additional testing and alters treatment. Genotypes generated by standard protocols used in forensic analysis (Beckman Coulter CEQ 8000 and ID kit) were supplied by Dartmouth School of Medicine for comparison to dpFRET STR genotyping. Samples were analyzed using dpFRET as previously described for the TPOX locus and results compared to current accepted protocols.RESULTS

Synthetic SNP Testing—Template Variation.

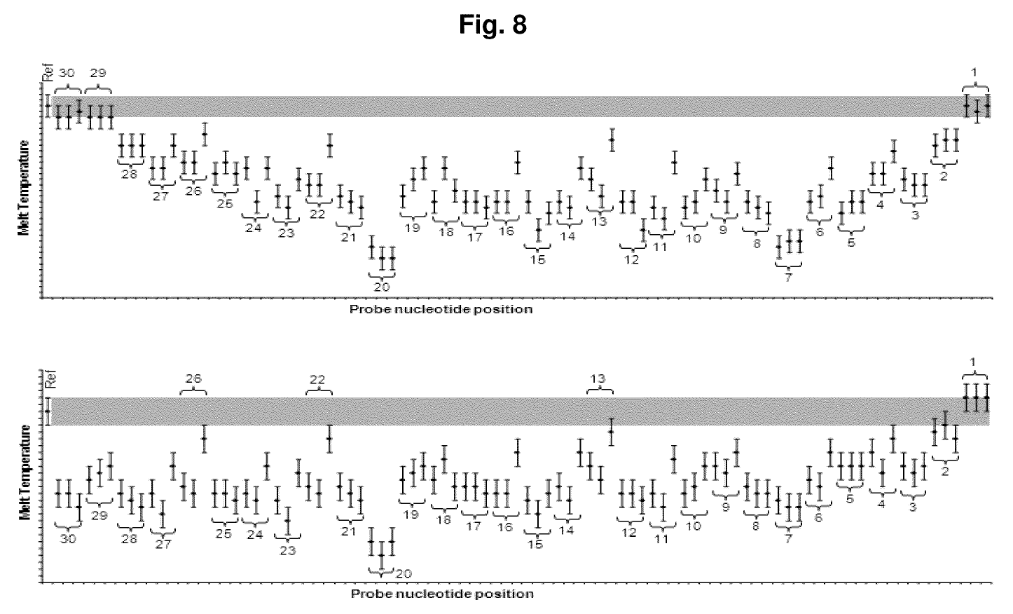

Results for the variable human sequence template library testing using a 30 bp probe are shown in FIG. 8. dpFRET results are shown in the top panel which depicts the melt temperature for each positional change within the template tested with a fluorophore labeled probe. Error bars of ±0.4° C. are labeled for each data point to account for thermal block variation. The range for an exact match (reference template) is highlighted across the graph. The bottom panel represents similar testing with an unlabeled probe (standard intercalating dye melt analysis) to explore fluorophore effect on melting temperature. The 30 bp 3′ fluorophore labeled probe resulted in discrimination of any change at any position except for mutations in the template complementary to probe nucleotides 30, 29, and 1. In contrast, the unlabeled probe was unable to discriminate mutations at multiple positions both distal and internal within the template (probe nucleotides 26, 22, 13 and 1). It is also important to note that the melt point graph is similar between labeled and unlabeled probes with the labeled probe displaying more significant variation from the reference for most points.

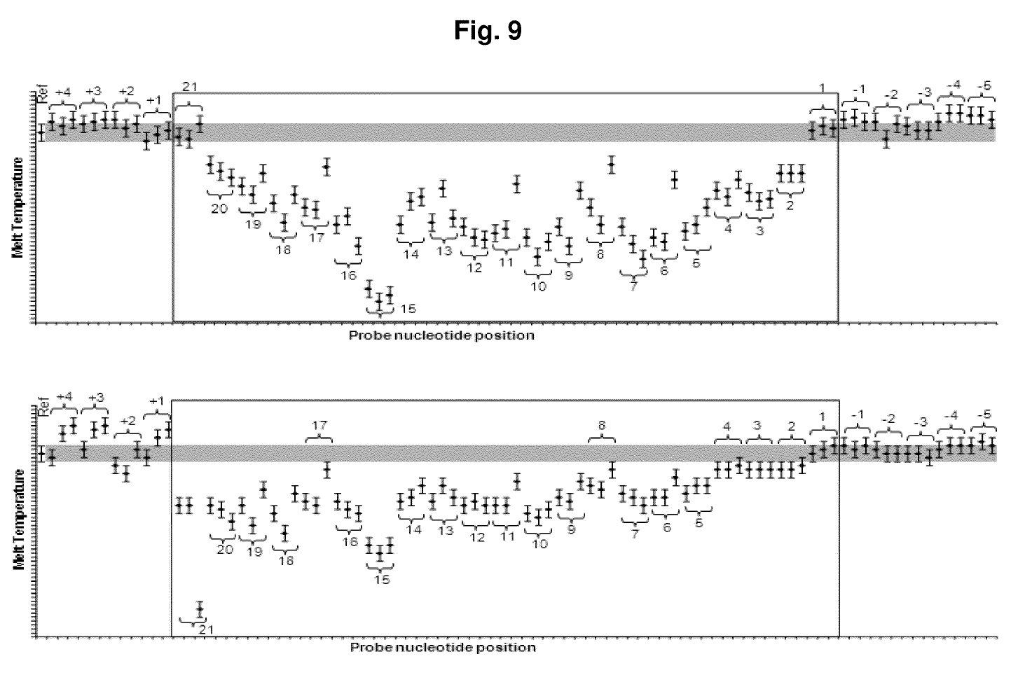

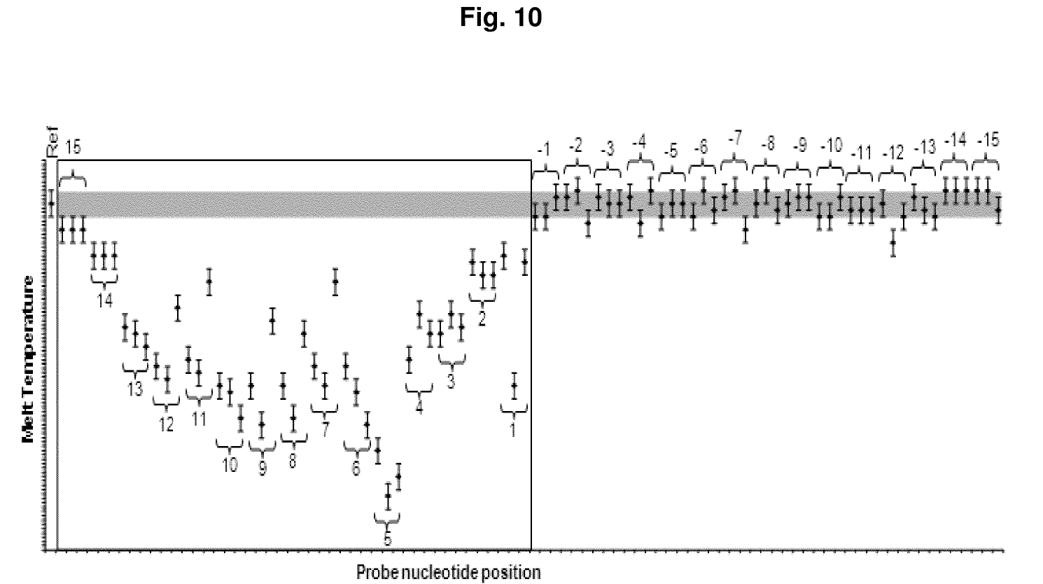

To understand effect of probe size, 21 and 15 bp fluorophore labeled probes were also tested and showed similar results with finer resolution at the ends of the template using the dpFRET approach. The fluorophore labeled 21 bp probe (FIG. 9 top panel) was indistinguishable from the reference for template mutations complementary to probe positions 21 and 1 and showed no effect due to template mutation in flanking sequence. Similar melting protocols using an unlabeled probe (FIG. 9 bottom panel) resulted in melt temperatures indistinguishable from the reference for mutations at multiple positions (probe nucleotides 17, 8, 4, 3, 2 and 1). Additionally, an effect was seen for mutations in upstream sequence flanking the unlabeled probe (probe nucleotides +1, +3 and +4). The fluorophore labeled 15 bp probe (FIG. 10) resulted in differential melt temperatures from the reference for all mutations except probe nucleotide 15 with a minor difference due to a flanking mutation (probe nucleotide −12). An unlabeled 15 bp probe was not tested.

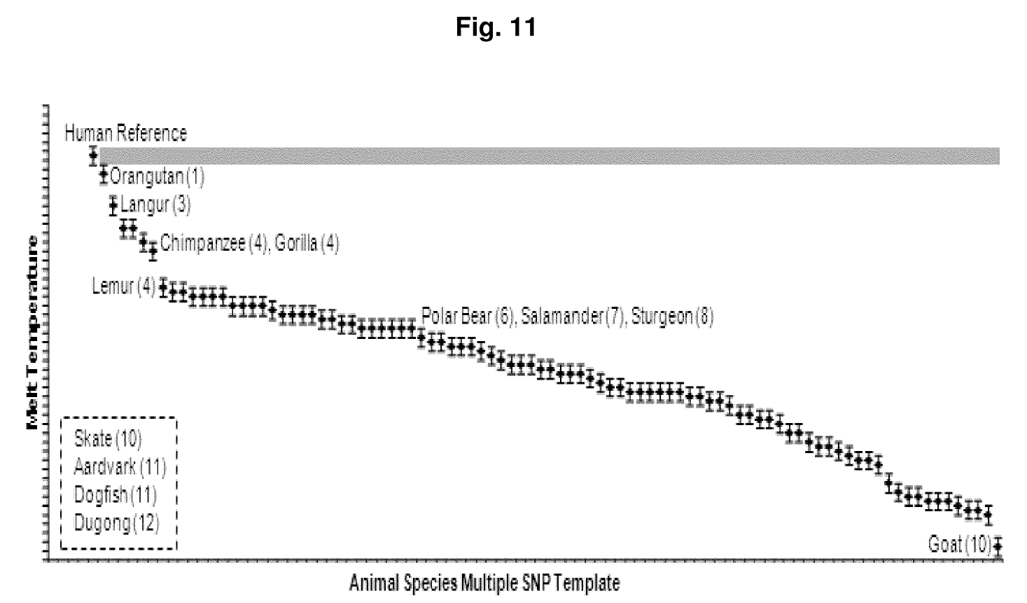

Synthetic SNP Testing—Species Variation. All synthetic animal species templates showed reduced melt temperatures compared to the human reference sequence when hybridized with a human probe sequence (FIG. 11). A few templates are listed with the number of SNPs in parenthesis to illustrate the range of sequence divergence. In general, increased number of SNPs within the template tended to reduce the melt temperature as would be expected. Four species templates (Skate, Aardvark, Dogfish and Dugong) did not produce melt curves when tested with a human probe sequence. All these templates had >10 SNPs. It should also be noted that closely related Orangutan sequence showed a differential melt temperature and contained only a single SNP. Unlabeled probes were not tested against the animal species library.

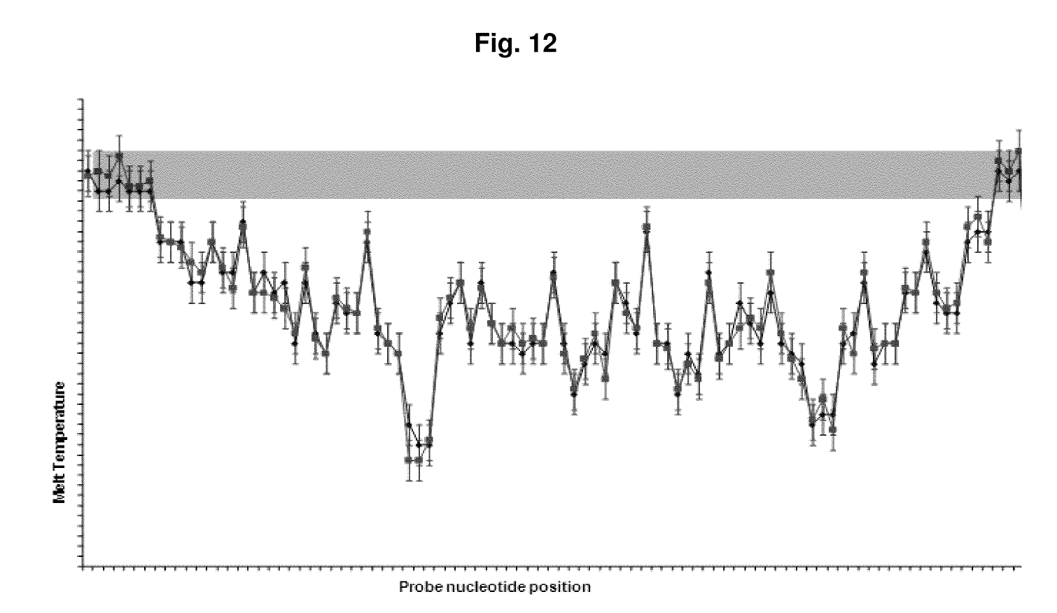

tDt Probe Labeling. Results for 3′ fluorophore tDt labeling of a 30 bp oligonucleotide probe showed no significant differences from a commercially synthesized probe (FIG. 12). Melt temperatures for both the commercially synthesized probe and tDt labeled probe were within ±0.4° C. for each template within the human variation library. Labeling efficiency of the enzyme was extremely low and did not provide significant amounts of reagent for sample testing.

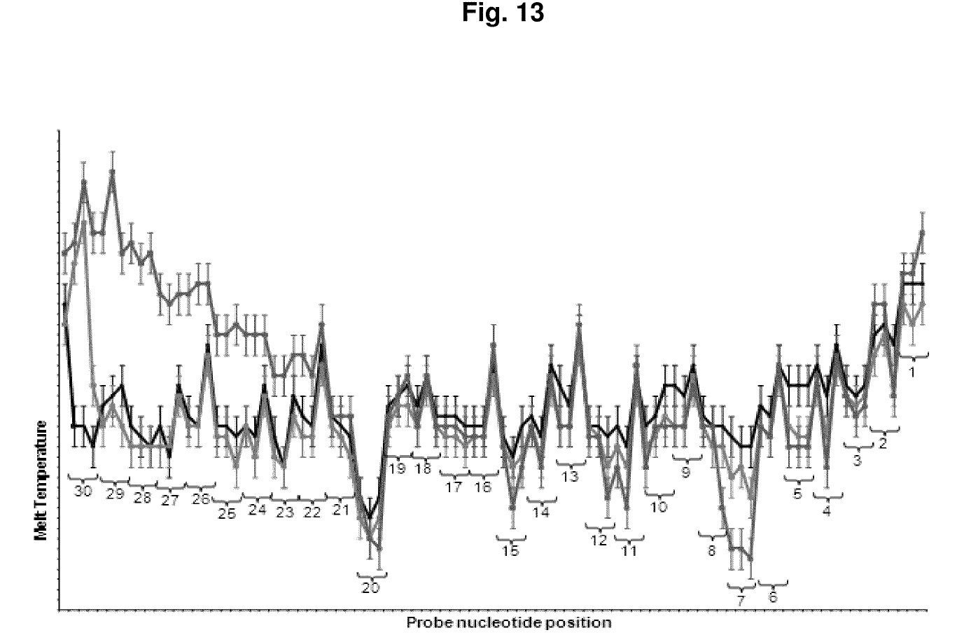

Effect of Inosine on Probe Hybridization. Addition of inosine at variable positions had a significant effect on probe melting relative to mutations at each position in the template (FIG. 13). In order to make comparisons, all melt temperatures were increased by 2° C. for the single inosine probe and by 6° C. for the triple inosine probe. The probe treatment with one inosine at the 3′ (position 30) end of the probe showed a significant difference from an unmodified probe for all three mutations at position 30 of the template with only a slight difference at position 5 downstream of the modified residue. The probe treatment with inosine at positions 30, 29 and 28 showed a significant difference from the unmodified probe at positions complementary to the inosine residues, at positions adjacent (positions 27, 26, 25 and 24) and at positions distal (positions 7 and 5) to the modified residues.

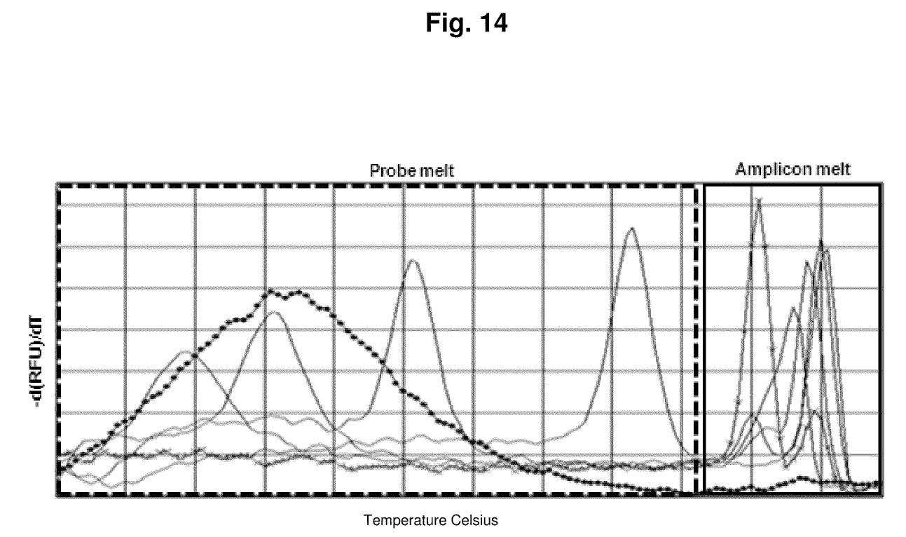

Haploid Locus Testing (Cytochrome B). Results for species testing from a limited number of species is shown in FIG. 14 and listed in Table 2. All species tested were positive for probe hybridization except python which differed by >10 nucleotides from the human reference probe. The non-template control showed some non-specific probe signal but did not exhibit the characteristic amplicon positive peak indicating a negative result. All other samples resulted in an amplicon peak.

TABLE 2
Cytochrome B speciation melt temperatures

| ID | Amplicon | Probe |
|---|---|---|
| Human 1 | 85.0 | 71.5 |
| Human 2 | 85.5 | 71.5 |
| HumPeng 1 | 85.5 | 46.0 |
| HumPeng 2 | 85.5 | 45.5 |
| Flamingo 1 | 84.0 | 39.5 |
| Flamingo 2 | 84.0 | 39.5 |
| Python 1 | 80.5 | — |
| Python 2 | 81.0 | — |
| Ferret 2 | 83.0 | 55.5 |
| Negative | — | 47.0 |

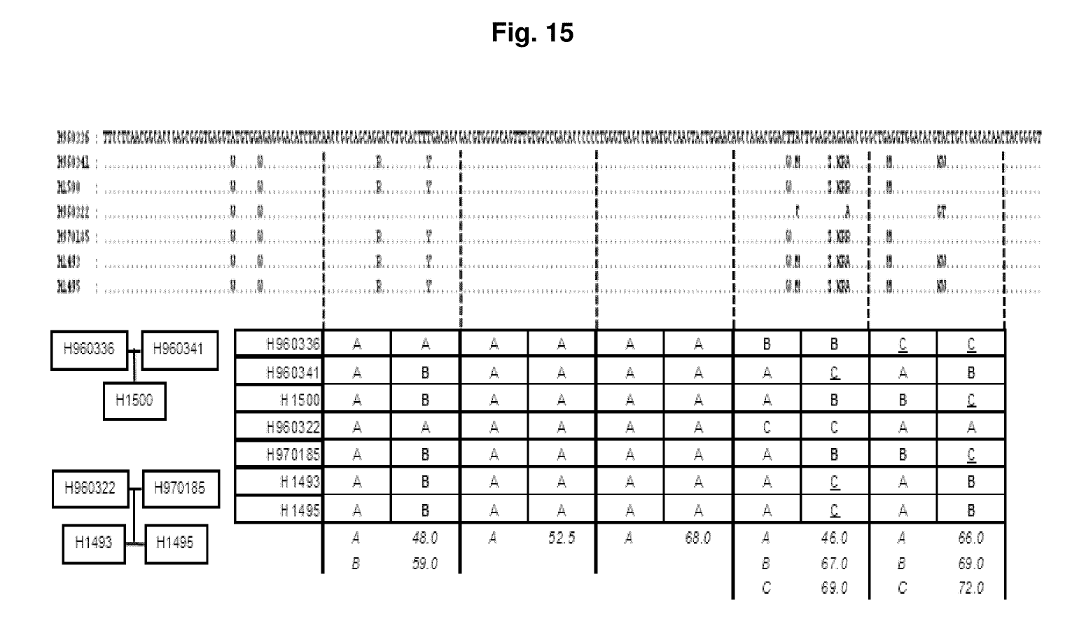

Diploid Locus Testing (MHCdrBeta). Paternity results for real world testing of two known Humboldt Penguin families are shown in FIG. 15. A sequence alignment for the amplification products produced using the universal Mhc DRB PCR assay is listed at top of the figure. Differences relative to sequence for the H960336 individual are listed using standard degenerate nucleotide base codes (i.e., Y=C or T, R=A or G, etc.). All melt temperatures generated by dpFRET analysis were converted to allele designations of either A, B or C for presentation purposes. Paternity results previously established by Brookfield Zoo through both Southern blot analysis and zoo keeper records for the two families are depicted at the bottom of the figure. Previously established paternity agreed with results generated by dpFRET analysis.

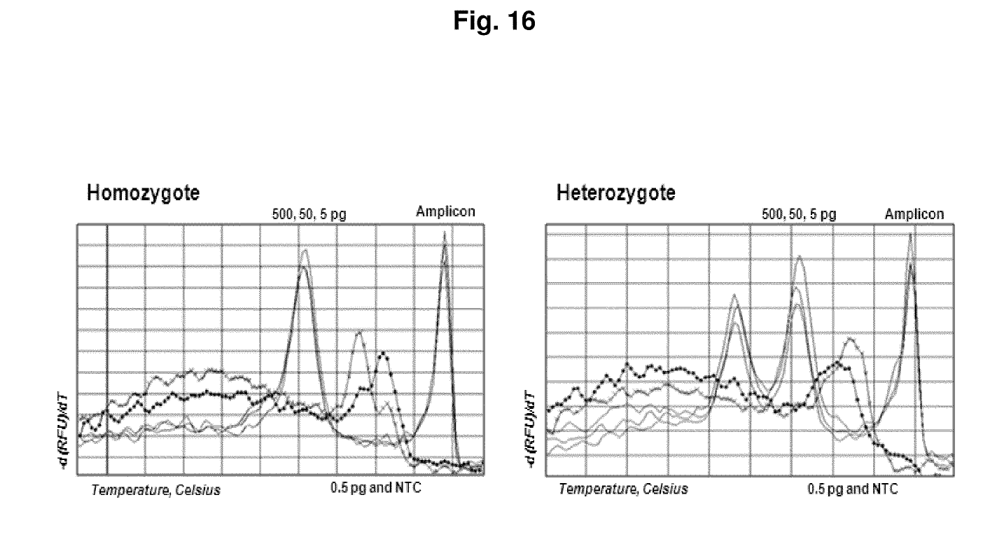

SNP Assay Sensitivity. The limit of detection using dpFRET for SNP analysis was 5 picograms (approximately 1 genome equivalent) for both homozygote and heterozygote samples (FIG. 16). Fluorescent signal showed no decrease for less concentrated samples and no allelic dropout was observed for the heterozygote. Both 500 femtograms (approximately 0.1 genome equivalents) and the no template control showed non-specific probe interaction as evidenced by a broad probe melt peak with neither sample resulting in a peak indicative of specific target amplification.

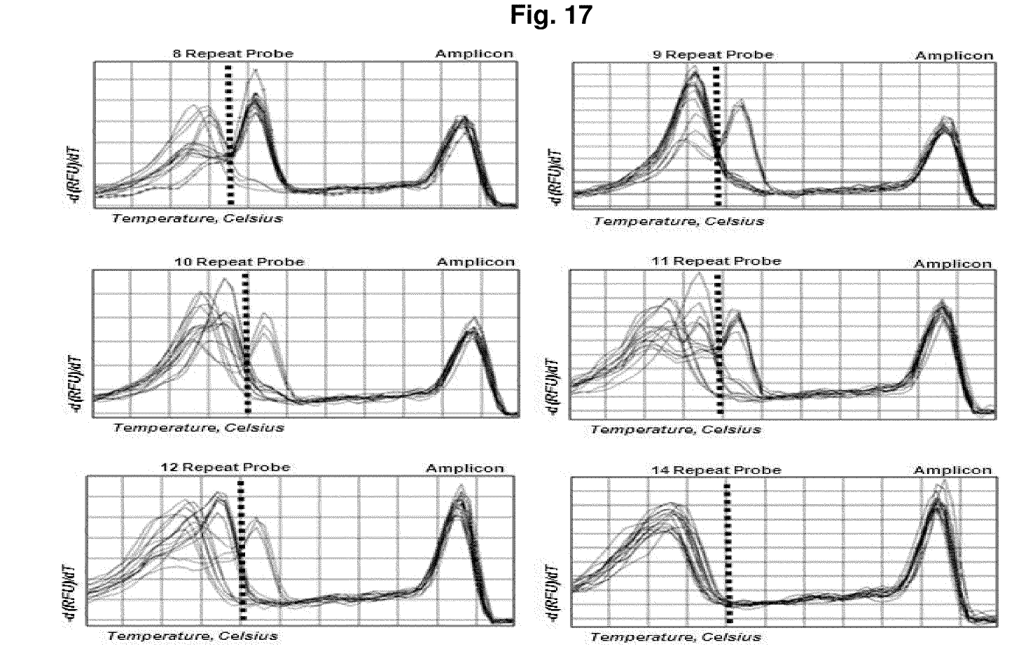

Microsatellite Locus Testing—Simple Locus (TPOX). dpFRET analysis of the TPOX locus for samples provided by the Johnson County Criminalistics Laboratory showed identical results to genotype data previously generated by the crime lab using standard capillary electrophoresis detection (FIG. 17). dpFRET melt curves for each allelic probe are shown.

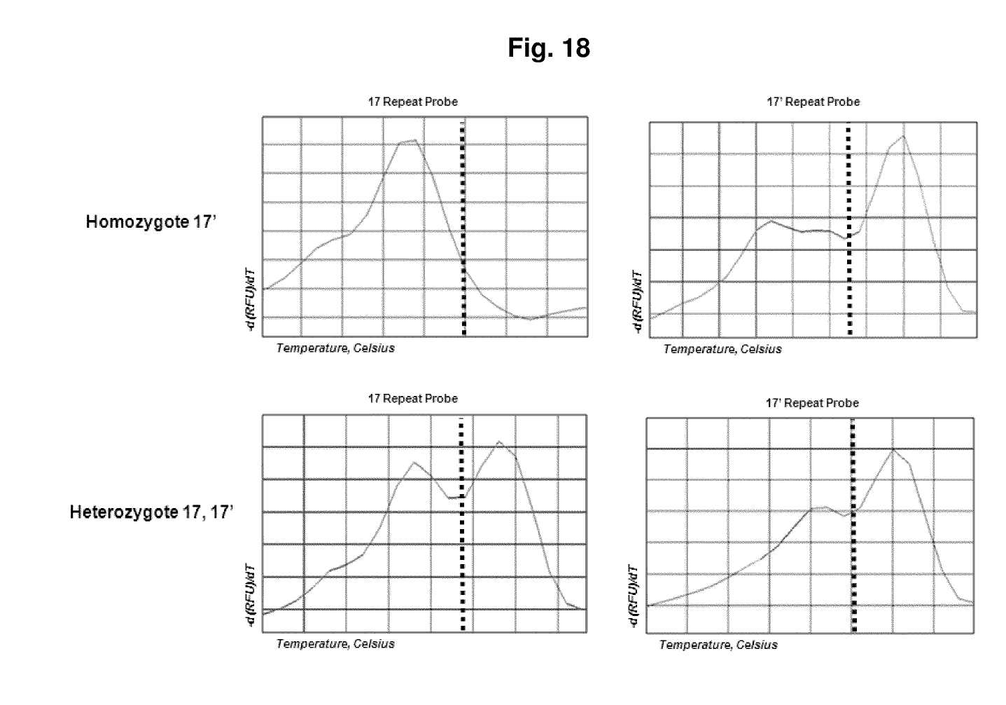

Microsatellite Locus Testing—Complex Locus (D3S1358). Similar to STR simple locus testing, dpFRET analysis of the D3S1358 STR complex locus resulted in similar although not identical results. When analyzed by size, complex STR loci can result in the same size profile for alleles that do not contain the same sequence. This is due an equivalent change (an addition to one core repeat with a deletion in the second core repeat) that cannot be differentiated based on size. Discrepancies for some samples were seen when analyzed by dpFRET due to the sequence based analysis of the approach that was able to detect this type of difference between alleles. As this complicated the comparison between dpFRET and standard approaches, an example of the results generated by dpFRET are provided in FIG. 18 to illustrate this potential phenomenon. Two individuals both typed as homozygotes and containing 17 repeats resulted in differential patterns (17′ homozygote and 17, 17′ heterozygote) when analyzed by dpFRET

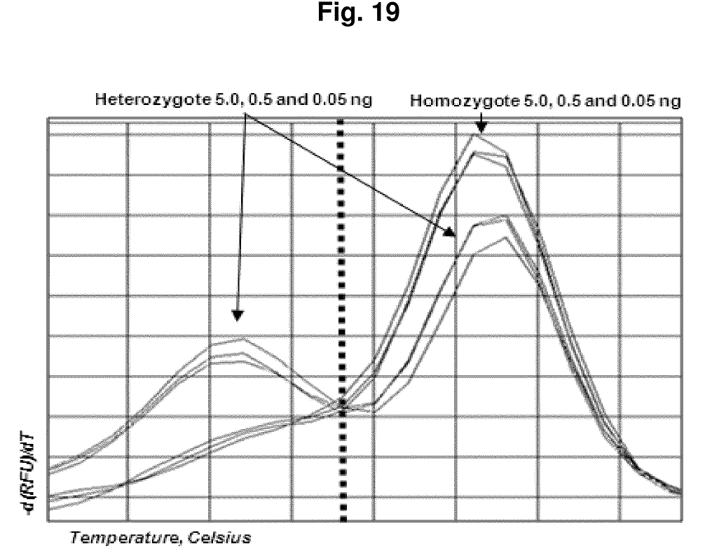

Microsatellite—Sensitivity and Lack of Allelic Dropout. Preliminary results to determine the limit of detection using dpFRET for STR analysis was 50 picograms (approximately equivalent to 10 genomic copies) for both homozygote and heterozygote samples (FIG. 19). It is important to note that fluorescent signal showed no decrease for less concentrated samples and no allelic dropout was observed for the heterozygote.

Microsatellite—Mixed Samples.

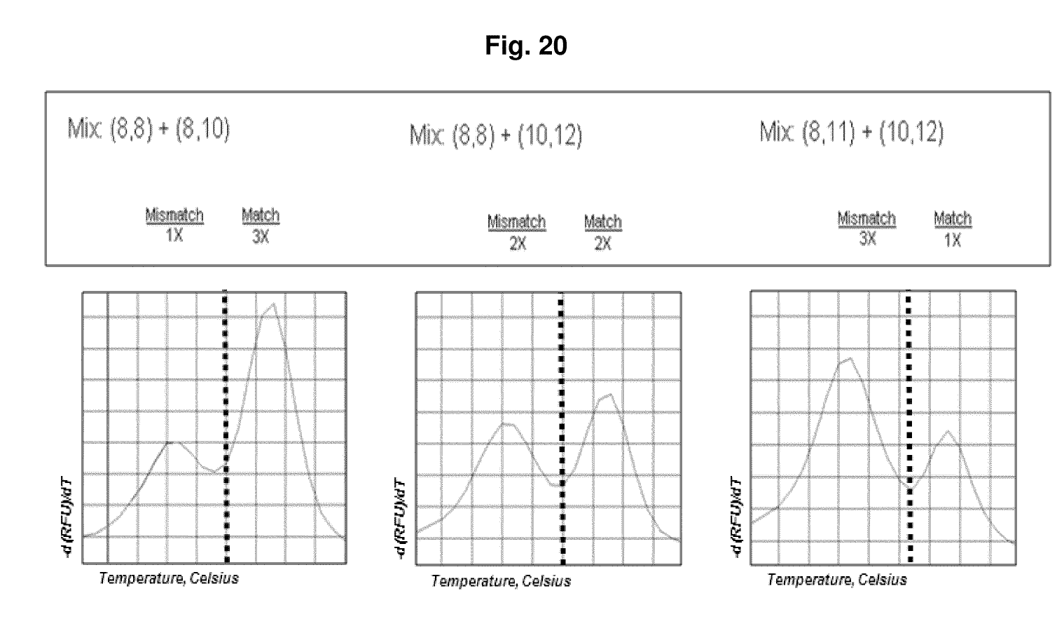

Artificial Mix. Artificial mixtures of homozygote and heterozygote samples tested with an 8 repeat allelic probe resulted in fluorescent match and mismatch signal intensity changes approximately equivalent to the concentration of allele within the sample (FIG. 20). The first mix composed of a homozygote and heterozygote (left panel) contained approximately 3× the amount of target allele (8 repeats) compared to non-target allele (10 repeats) and resulted in a significantly higher match peak signal intensity. It should be noted that the match and mismatch peak fluorescent intensities are not directly correlated with sample allelic content (match ˜170 RFU, mismatch ˜80 RFU). The second mix (middle panel) contained an equal proportion of target and non-target allele and resulted in approximately equivalent fluorescent intensities for the match (˜110 RFU) and mismatch (˜90 RFU) peaks. The third mix (right panel) was composed of 3× non-target allele and resulted in markedly higher mismatch peak signal intensity. Similar to the first treatment, peak height intensity did not correlate with sample allelic content (match ˜90 RFU, mismatch ˜130 RFU).

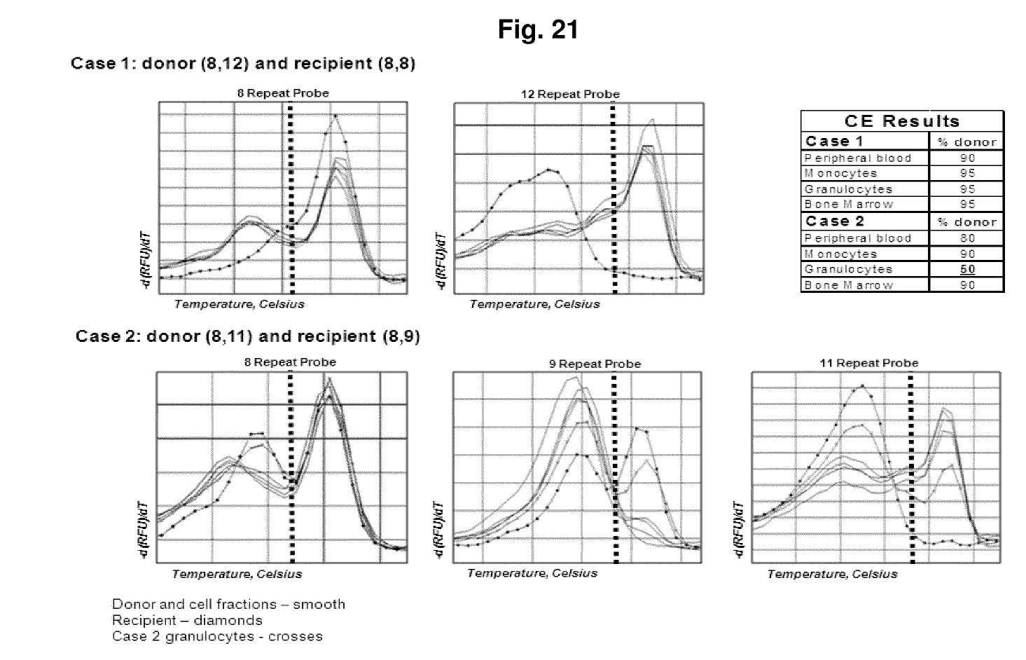

Bone Marrow Transplant Samples. dpFRET analysis for samples from two bone marrow transplant cases provided results similar to analysis by capillary electrophoresis (FIG. 21). Case 1 (top panel) resulted in all cellular fractions displaying donor genotype for both alleles (8 and 12) tested. This was in agreement with results generated by capillary electrophoresis that detected 90-95% donor for all fractions. dpFRET testing for case 2 (bottom panel) resulted in donor genotype for all cellular fractions except granulocytes which showed a mix of both donor and recipient at approximately a 1:1 ratio. This result was in agreement with previous capillary-based testing that showed a 50% contingent of donor genotype within this sample. Additional cases were tested (data not shown) and showed similar results to Case 1. Additionally, all blinded donor and recipient allelic assignments generated by dpFRET analysis were in agreement with previously established genotypes.

### DISCUSSION

Hybridization-based genotyping of changes in DNA often depend on oligonucleotide melting temperature (Tₘ). The Tₘ of duplex DNA is defined as the temperature where one-half of the nucleotides are paired and one-half are unpaired. Tₘ can be predicted using a variety of formulas with the most accurate being the thermodynamic nearest neighbor model. The nearest neighbor model is based on the assumption that probe hybridization energy can be calculated from enthalpy and entropy of all nearest neighbor pairs, including a contribution from each dangling end. Dangling ends account for the effects seen when a shorter probe is bound to a target with flanking sequence. Various interactions contribute to probe/template stability, but it has been demonstrated that melting of the complex is initiated at the ends of the duplex. It is this dangling end effect that provides dpFRET with a higher level of resolution as compared to an intercalating dye. The difference is derived from the preference of melting to initiate from the ends of the duplex. This is commonly referred to as end fraying or end effects and can propagate several base pairs into the duplex. The goal of synthetic testing for SNP genotyping was to determine optimal probe design and performance limitations.

The first phase of the development of dpFRET for SNP genotyping involved determination of the effect of probe size on resolution. Initial testing used a synthetic library of templates that encompassed any potential change at every position complementary to the probe sequence. The most obvious result for all probe sizes tested (30, 21 or 15 bp) showed that this approach is capable of producing a differential melt relative to a perfect match with the probe sequence. In other words, a mutation at two different locations within the sequence can potentially produce the same melt temperature, but that temperature is always lower than a perfect match between the probe and reference sequence. Changes at the ends (5′ and 3′) of the template were indistinguishable from the reference sequence for larger (30 and 21 bp) probes most likely due to inadequate end effects. A reduction in the size of the probe (15 bp) produced a differential melt temperature for all changes.

The most likely explanation for the effect of higher resolution with a reduction in probe size is a decrease in the amount of energy required to break the bonds between the probe and template. A smaller oligonucleotide requires less energy and a base mismatch will therefore have a more intense effect on melting temperatures of smaller sequences. It is also likely that end effects are amplified proportionally with decreasing probe size. In its current state, dpFRET can be applied for SNP discovery with follow-on sequencing for determination of the exact position and mutation. For purposes of SNP screening, it may be necessary to take into account design considerations for discrimination of certain targeted changes. Overall, probe size should be limited to 15-30 bp depending on the particular application desired.

For both the 30 and 21 bp probes, dpFRET showed higher resolution for internal template changes than SYBR Green I (intercalating dye) alone. This result lends credibility to the hypothesized end-effects theory. Internal mismatches are averaged out across the template as it melts when utilizing an intercalating dye. Any single mismatch is averaged with all matching nucleotides across a template producing a lower signal to noise ratio. By localization of differential melting signal to the end of the hybrid complex, the effect is more significant because FRET can only occur across a limited distance. So, signal differences contributed from the mismatch remains constant, but the noise produced by dye intercalated at a distance is minimized. This same approach can be used for other applications with limited SNPs including a range of synthetic template sequences.

The limits of resolution for multiple SNPs within a template sequence were also tested. Many other hybridization-based genotyping systems are unable to genotype more than a single SNP per assay design. One of the benefits of the inventive dpFRET method is the ability to detect multiple changes within one template with a single assay design. To test the limits of this approach, a template library was synthetically generated that encompassed one to twelve SNPs in varying configurations based on a region of Cytochrome B sequence. The reference and complementary probe sequence were based on human Cytochrome B with the intended application for animal species genotyping.

As many as nine collective mutations within a 30 bp sequence were detected. Beyond nine base pairs, the probe and template were not able to hybridize in a manner sufficient to intercalate dye and donate signal to the fluorophore probe for genotyping. Hence, even with 30% divergence between the probe and template, a signal was generated. Similar to probe size testing on the template variation library, all probe/template complexes showed a reduced melt temperature compared to the reference human sequence but were unable to classify all templates as unique. This is most likely due to the fact that multiple mutations at variable positions can have the same destabilizing effect on the DNA duplex and would not therefore produce a unique melt temperature.

DeoxyInosine (dI) is a naturally occurring base that, while not truly universal, is less destabilizing than mismatches involving the four standard bases. Hydrogen bond interactions between dI and dA, dG, dC and dT are weak and unequal with the result that some base-pairing bias does exist with dI:dC>dI:dA>dI:dG>dI:dT. It is believed that this base pairing bias would differentially affect melting behavior of the whole complex. In other words, a mutation from C to T at one position would bind inosine in a weaker manner and affect the melting of the nearest neighbors. Incorporation of inosines at the end was most likely to show this effect. Results demonstrated that single and multiple insertions of inosine within the probe sequence were able to alter the melting behavior of corresponding template mutations and nearest neighbor mutations. This effect has been modeled and can alter the melting behavior of a probe in different ways based on number and location of inosine bases within the probe, probe sequence and template nearest neighbor sequence. By locating inosine bases in a sequence dependent fashion, a unique temperature for any change within a template may be provided.

Terminal transferase (TdT) is a template independent polymerase that catalyzes the addition of deoxynucleotides to the 3′ hydroxyl terminus of DNA molecules. TdT can be used to incorporate a fluorophore labeled nucleotide at the 3′ end of an oligonucleotide probe. The FRET system that was tested used Texas Red as the acceptor fluorophore that was incorporated by tDt as a dUTP. Unfortunately, tDt labeling has been shown to be extremely inefficient at incorporation of this particular fluorophore. This is most likely due to interference with the active site of the enzyme. Additional end labeling strategies (ULYSIS) were tested and proved unsuccessful. Future directions for probe generation would include testing of alternative fluorophores with tDt.

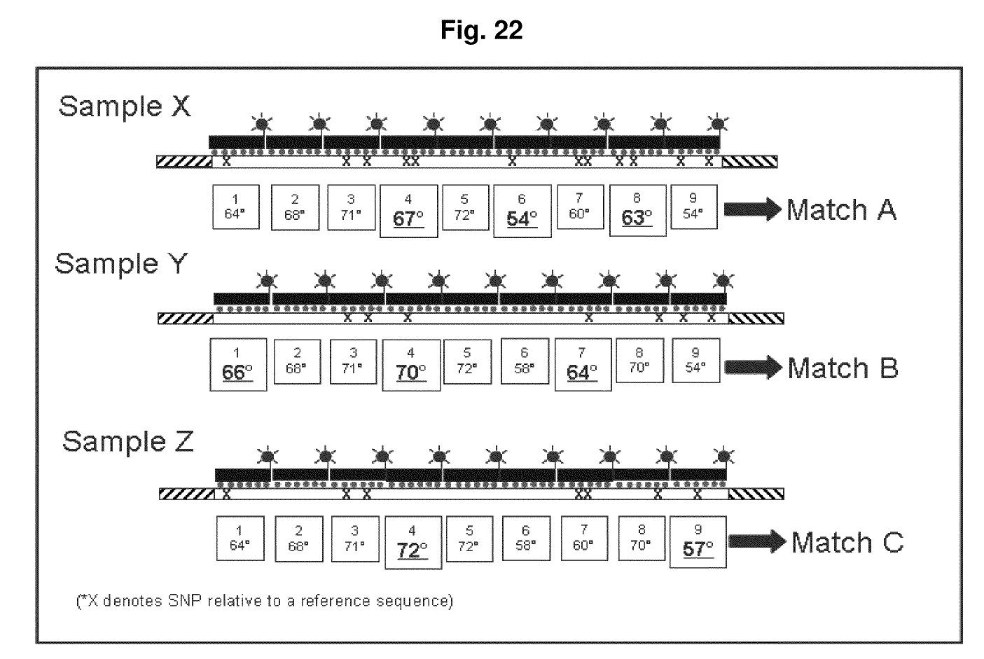

As shown in FIG. 22, the relative sequencing methods of the present invention enable one probe set to be designed against a reference sequence. Each probe would encompass approximately 30 base pairs of sequence and would stretch across the sequence of interest. For example, if one were interested in looking for SNPs in 270 base pairs of human mitochondrial Dloop (control region) sequence, 9 probes would be designed that covered the region of interest. Multiple samples could then be tested with each probe to produce a melt temperature either matching or lower than the reference sequence. Any probes that produced a lower Tₘ would signify the presence of a SNP relative to the reference sequence at that probe position. With the current state of dpFRET, follow-on sequencing would be needed to identify the exact mutation and position of the SNP. In the case of human forensics, the reference sequence would be represented by the victim and the samples would be represented by potential perpetrators. The benefit to this approach would be the ability to screen a multitude of potential samples at a significantly reduced cost compared to standard sequencing. All samples matching the victim could be disregarded and the focus could be placed on probative samples. A similar approach would also be useful for screening large numbers of clinical samples for either SNP discovery (i.e., a change in a gene promoter) or screening following identification of a candidate SNP.

“Real World” Testing and Development. Synthetic testing was used to define the limits (probe size, assay optimization, etc.) of dpFRET SNP genotyping, but practical application would involve amplification of a target sequence. Initial tests used a haploid marker (Cytochrome B—mitochondria) to minimize melt curve complexity (single peak). This was followed by a diploid marker (MHCdrBeta) testing to explore the ability of the assay to discriminate two different alleles within the same individual. Both assays consisted of unique primer designs that were based on alignments of published sequence for multiple species. Testing has shown both assays to be successful for amplification of multiple species and could have potential utility in a number of applications for species and individual identification. It is also important to note that a single reference (human) was used to design probes for testing of both markers. This highlights the broad applicability of this approach.

The results from CytB testing showed that the melt peaks for the amplicons were not able to resolve all species and showed limited resolution (5° C.). The probe melt peaks on the other hand resolved all species tested and showed a much higher level of peak resolution (32° C.). This result agreed with results from synthetic testing and is due to the ability of dpFRET probes to resolve divergent sequences. In a similar fashion to synthetic testing, the python sample showed no probe peak due to sequence divergence beyond the 10 bp or 30% limit. The no template control (NTC) resulted in a broad probe melt peak but no amplicon peak. This phenomenon has been reproduced in follow-on development and is primarily due to excess probe concentration. The probe can form a probe/probe dimer that produces a signal at a significantly reduced melt temperature without producing an amplicon peak. Optimization of probe concentration can alleviate this effect for most probes, but is not really necessary due to the absence of an amplicon peak. Due to strong SYBR signal, the amplicon can produce a signal and is used as a qualification of positive amplification. Without the presence of an amplicon peak, the probe/probe dimer signal can be classified as noise. Finally, it is important to note that the amplicon melt peaks included a small shoulder peak. It was discovered through follow-on development and testing that this shoulder was due to a minor population of unlabeled probe that results from incomplete synthesis that allows the unlabeled probe to participate in the amplification. By adding a small amount of EDTA with the probe post-amplification, this anomaly was removed. This is due to the fact that EDTA at the proper concentration can chelate magnesium required by the polymerase enzyme as a cofactor.

The MHCdrBeta marker has been used previously in many studies for individual identification and paternity analysis. A universal assay for application in many species has yet to be described. Following assay development using a number of potential primers, a pair was optimized that was capable of producing a product in a number of species. Initial testing used human sequence as a reference. The same probes were applied to Humboldt penguin samples provided by Brookfield Zoo that had been tested previously for paternity using Southern Blot hybridization (Brookfield, Ill.). As this can be a labor intensive process, dpFRET was explored as an alternative using previously designed human probes. The probes were not only able to hybridize to penguin sequences, but were able to resolve differences between individuals. Examination of the sequence showed that although differential SNPs from the human design existed that were conserved among all penguins tested, differences between individuals could still be resolved. For regions of the amplicon that were more highly divergent from human, probes were designed against a Humboldt reference sequence which showed better resolution of heterozygote alleles. Thus, the methods of the present invention provide the ability to resolve multiple alleles (heterozygotes) within a single individual and a further demonstration of the flexibility of the approach and flexibility of assay design. This is particularly important in fields like conservation biology where studies require designing assays specific to each and every animal tested. The dpFRET approach is not only flexible enough to applied across a range of species for paternity and population studies, but requires significantly less resources than the current approaches.

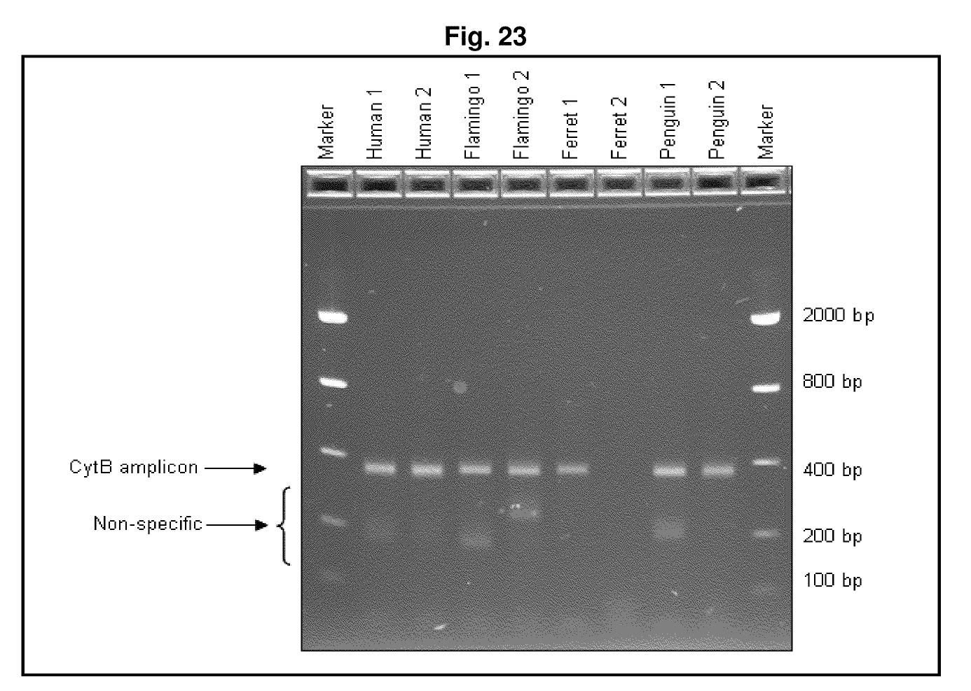

For application of dpFRET in fields with limited amounts of sample, it is important to resolve the sensitivity of the inventive methods. Human testing with previously quantified genomic material showed an initial detection limit of a single copy. This result is not surprising due to the fact that dpFRET uses 50-80 cycles of amplification depending on the approach. This level of sensitivity has already been shown for approaches using Taqman detection and is primarily due to the fact that non-specific product produced with high numbers of amplification cycles is not detected due to signal generation produced by probe hybridization. dpFRET is capable of capitalizing on the same strategy. Only non-specific product with less than 30% divergence will produce a signal with dpFRET. An example of product generated for CytB in different species is shown in FIG. 23. In addition to specific product at 350 bp, multiple non-specific products are also amplified that show no signal upon detection and genotyping with dpFRET. These results are significant for fields like forensics and clinical testing wherein sensitive detection is required and target concentration is typically quite limited.

Results from SNP testing using dpFRET shows that the inventive methods are robust for detection of few copies within a sample. It is also successful at genotyping both haploid and diploid loci with no impact on detection of multiple alleles. Design strategies are highly flexible and are capable of detecting single or multiple SNPs using a single assay. Potentials for development include increased discrimination between mutations and reduced reagent costs through alternative approaches.

dpFRET Microsatellite Testing and Development. Repetitive sequences can be referred to as microsatellite, short tandem repeat, variable number tandem repeat and a host of other terms. The concept behind the application of these markers is that a genotype can be generated based on the number of repetitive core sequences. The greatest strength to these markers is their ability to produce multiple alleles per assay providing more information per test than biallelic SNPs. Microsatellites or STRs are accepted as the marker of choice for forensics and ecology and are more recently being valued in clinical studies for the ability to monitor progression of cancer and aid in monitoring transplant success. Information is typically generated by amplification followed by sizing of the alleles by capillary electrophoresis. Not only is this approach subject to a number of artifacts, but also requires specialized equipment and a high degree of training to generate genotypes. A simpler approach to interrogate these highly informative markers would provide a significant step in more routine application of this approach.

Microsatellite sequences can vary in content but typically have a similar structure. Conserved flanking sequences are used to amplify a repetitive region composed of a core repetitive section. This core repeat can be composed of either a single sequence (simple repeat) or multiple sequences (complex repeat) with additional SNPs potentially present within. With the success of dpFRET for SNP genotyping, the next step in development consisted of exploring application for microsatellite genotyping and how to apply dpFRET to repetitive sequences. A design approach was developed that would permit identification of a specific allele within a sample wherein the microsatellite locus was designated with three regions composed of a ‘reporter flank’, ‘core repeat region’ and ‘anchor flank’. It was hypothesized that the anchor flank could be designed with a higher Tm than the fluorophore labeled reporter flank. This would favor hybridization of the anchor region first, followed by hybridiztaiton of the core repeat region of the probe and assuming an exact match, this would be followed by the reporter flank.

Upon melting of a perfect match, a signal would be generated indicative of the presence of the number of repeats contained within the probe. If the probe were to encounter a mismatch with the template sequence, the result would be decreased hybridization with the reporter region of the probe resulting in decreased signal intensity and more importantly a lower melting temperature. This would primarily be due to the reduction in bonding energy of the probe due to imperfect hybridization and would result in generation of differential melting temperatures between a probe/template match or mismatch. A number of different designs were tested for varying lengths of both the reporter and anchor flanks. Shorter flanks resulted in partial peak separation. A Tm difference of approximately 10-15° C. between the reporter and flank proved to be successful for discriminating the presence of an allele within a sample. This was followed by extensive testing of both a simple and complex locus.

TPOX is located on chromosome 2 within intron 10 of human thyroid peroxidase gene. TPOX is one of the loci typically employed for individual identification as part of the collection of loci known as CODIS. Validated primer sequences from the Promega PowerPlex kit were used to remove any ambiguity generated by in-house designs. Following brief optimization for 80 cycle amplifications, probes were designed for common alleles (8-12 repeats). Samples provided by the Johnson County Crime Lab that were previously typed by CE were tested and showed agreement between CE and dpFRET genotypes. The same approach was then tested on a complex locus. D3S1358 is located on chromosome 3, is not known to be located within a coding region and is also one of the core loci within CODIS. Similar design and testing to that of the SNP design and testing discussed hereinabove were used and results were equally successful. This shows that dpFRET can be used with existing primer designs and simply acts as a new method of allele detection to replace size-based CE genotyping.

D3S1358 showed discordant results with CE generated genotypes. As CE is only able to differentiate differences in size, a complex locus with more than one core repeat has the potential to generate the same size product with different alleles. For example, D3 17 and 17′ are different alleles but cannot be differentiated by CE. Testing with dpFRET was able to differentiate these genotypes due to differential probe hybridization (individual 11). To further prove this hypothesis, amplification products could be cloned and sequenced to verify the presence of two alleles. Similar results were seen for 15 and 15′ alleles.

Allelic dropout is also an important consideration for analysis of trace level samples. This is primarily due to preferential amplification of one allele. For additional reasons, it is important to quantify starting material prior to CE based testing. The dpFRET approach to genotyping was tested for this effect and no significant allelic drop out was observed. In addition, varying amounts of starting material were tested and all samples showed equivalent results. This is primarily due to the difference in the approach to amplification. CE based typing typically utilizes 30-40 cycles. dpFRET uses 50-80 cycles of amplification. This provides the opportunity to amplify any target alleles that might be present and produces an equivalent signal. This is of particular importance in both forensic and clinical applications.

Both allelic drop out and pre-quantification are also important aspects in testing of mixed samples. dpFRET was tested for application to mixed samples and showed melt peak heights approximately equivalent to starting allele concentrations. This is evidenced by the fact that homozygotes routinely result in higher (typically double intensity) signal than heterozygotes for the match peak and lab generated mixes display peak heights equivalent to the concentration of alleles within the sample. dpFRET was also successful at reproducing donor and recipient percentages within a sample as compared to CE. Some variation was seen through testing most likely due to the amplification approach. The current protocol uses 40 cycles of double-stranded amplification followed by 40 cycles of single stranded amplification in a separate reaction. Using this approach, a single dsAMP is split across multiple ssAMPs followed by addition of allele specific probes to individual reactions. This minimizes the amount of sample required (sample is only added to the dsAMP) but also provides multiple opportunities for sampling error to introduce variability in peak height. Less variability is seen with closed tube asymmetric 80 cycle amplification protocols but requires multiple aliquots of sample for testing of each probe. This necessitated exploring other approaches for minimizing the need for testing with every allelic probe.

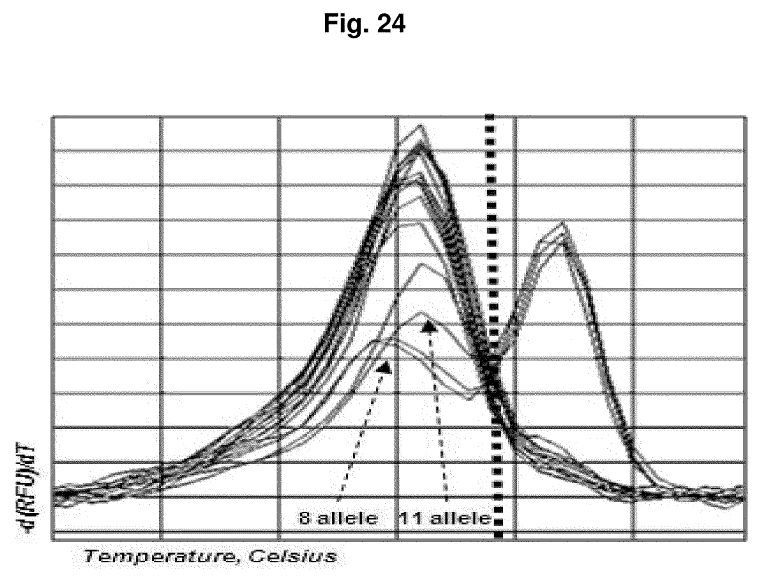

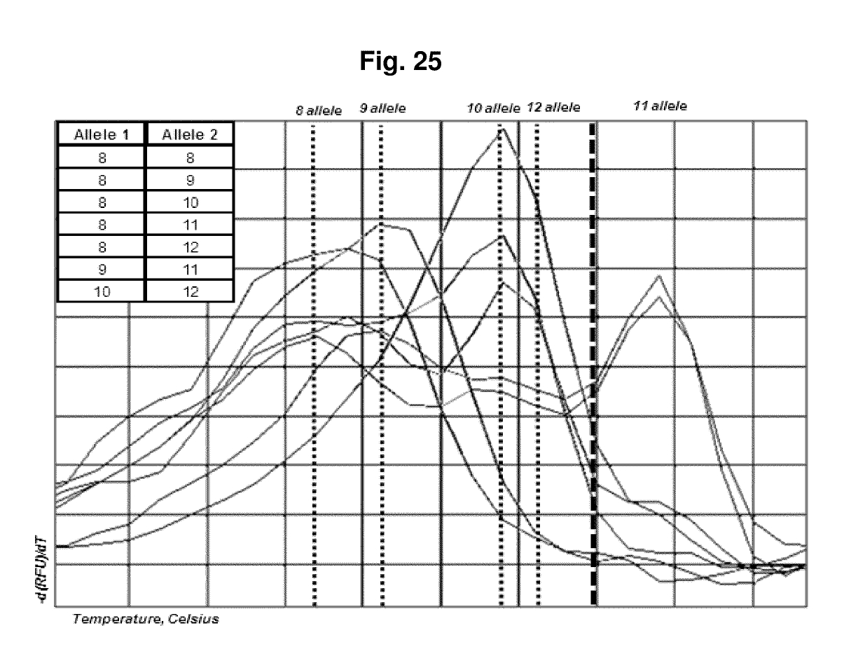

It was noticed early in testing that samples showed variation in the mismatch peak profile based on what mismatched allele was present. As seen in FIG. 24, four individuals all containing 9 repeat alleles showed a match peak. Two of the individuals contained an 8 repeat allele and two individuals contained an 11 repeat allele. This reproducibly resulted in differential melt curves for the mismatch allele. This phenomenon was further explored with other repeat probes. It was determined that higher number allelic probes had better discrimination of mismatched melt peaks. Testing with an 11 repeat probe (FIG. 25) was able to reliably differentiate the full allelic complement of a sample beyond a simple match/mismatch qualification. This is significant in that it provides a potential to genotype a sample using dpFRET and a minimal number of probes. Further development of the melt curve approach is needed for higher resolution.

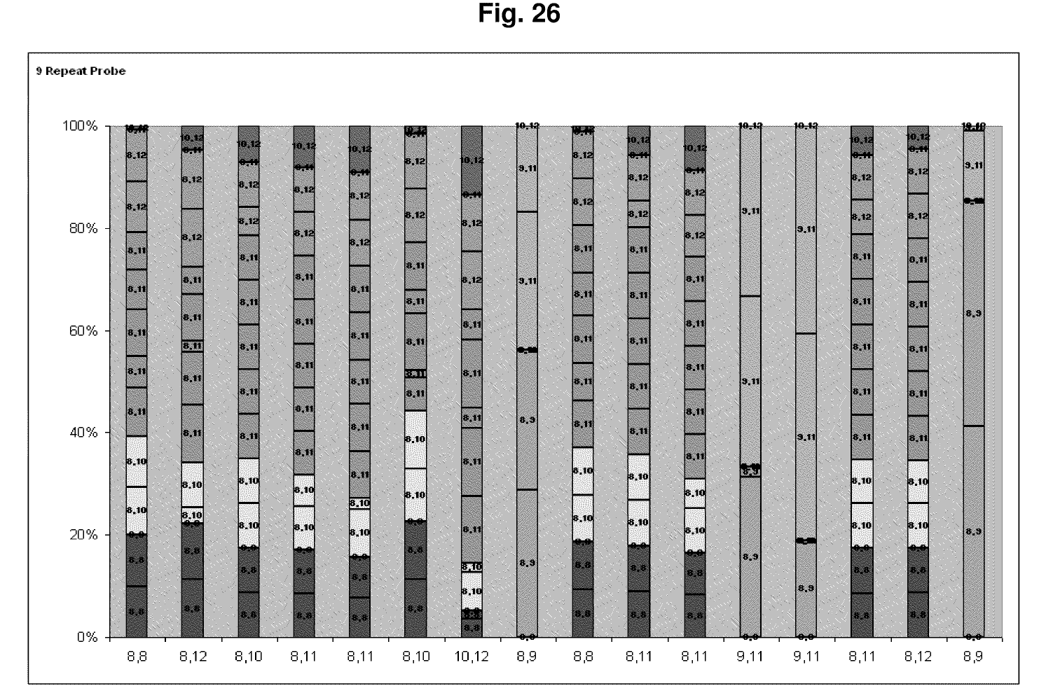

Utilizing the current melt curve approach there is also potential for higher resolution through analysis of melt curve shape. This requires a curve fitting approach. Using Chi-square analysis, each curve is compared against each other at each point to determine the difference between the observed and expected curve. Allelic calls for each sample are then made based on similarity. In the case of a 9 repeat probe (FIG. 26), both 8,9 and 9,11 individuals resembled each other but not any other individuals. This is primarily due to the presence of a match peak for the 9 repeat probe. The same samples were also analyzed for an 11 repeat probe and showed greater discrimination between patterns. Again, a higher level of resolution on the melt curve itself should provide for clearer allelic calls using a single probe.

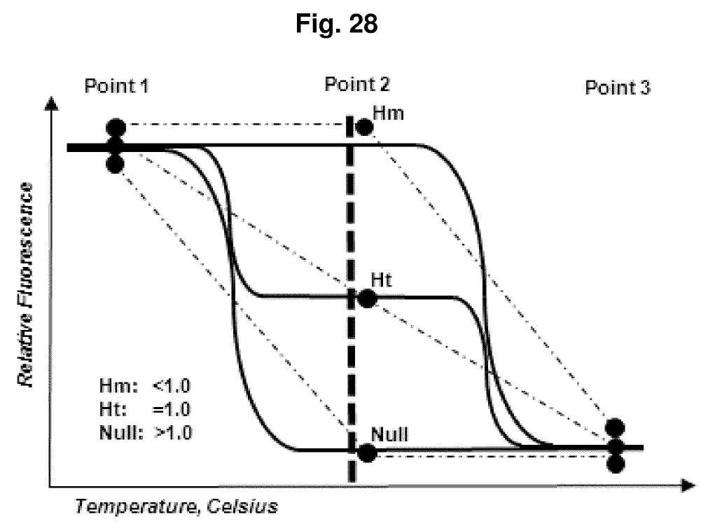

The initial approach to analysis requires additional time and analysis for melt curve generation. In an effort to reduce the time to results and simplify analysis protocols, discrete readings were taken at three points: 1) prior to probe/template denaturation; 2) a point midway between melting of a match and mismatched hybrid complex; and 3) following complete denaturation. By comparing the slope ratios of the points between time I and 2 and times 2 and 3, a quantitative method was established for genotyping of each probe. FIG. 28 is a graphical depiction of this approach. This same method can be applied for any probe as all the allelic probes can be measured at the same temperature (74° C.). By careful probe design, multiple markers can be analyzed under a single quantitative analysis scheme. This reduces the amount of data points required and significantly increases the speed of analysis.

Areas of Application for SNPs and Microsatelittes. The benefits to using dpFRET for discovering and screening changes in DNA are numerous. It is less costly than many other approaches to SNP and microsatellite genotyping due to the use of a single fluorophore. Probe design is extremely flexible and relatively sequence independent. Moreover, equipment requirements are minimal. Analysis is more objective than other approaches and is amendable to automation. Application of this new approach spans any field in need of looking at changes in DNA. For simplicity, examples of application to the fields of forensic science and clinical diagnostics will be detailed.

Forensic studies utilize a number of different marker types. Microsatellites have become the gold standard with increasing interest in using SNPs for nuclear, mitochondrial and Y chromosome markers. dpFRET provides the first opportunity to examine both SNP and microsatellite markers using the same equipment, chemistry and basic technical approach. This simplifies the needs of a forensic laboratory by reducing the required infrastructure. dpFRET analysis is also more rapid and less labor intensive than current approaches (i.e., CE). Also inherent in the approach is the ability to amplify the smallest possible microsatellite markers (“ultra miniSTRs”) because there is no requirement for differential marker size for CE separation. This has strong implications for genotyping of trace level degraded samples that could significantly advance studies in forensic science.

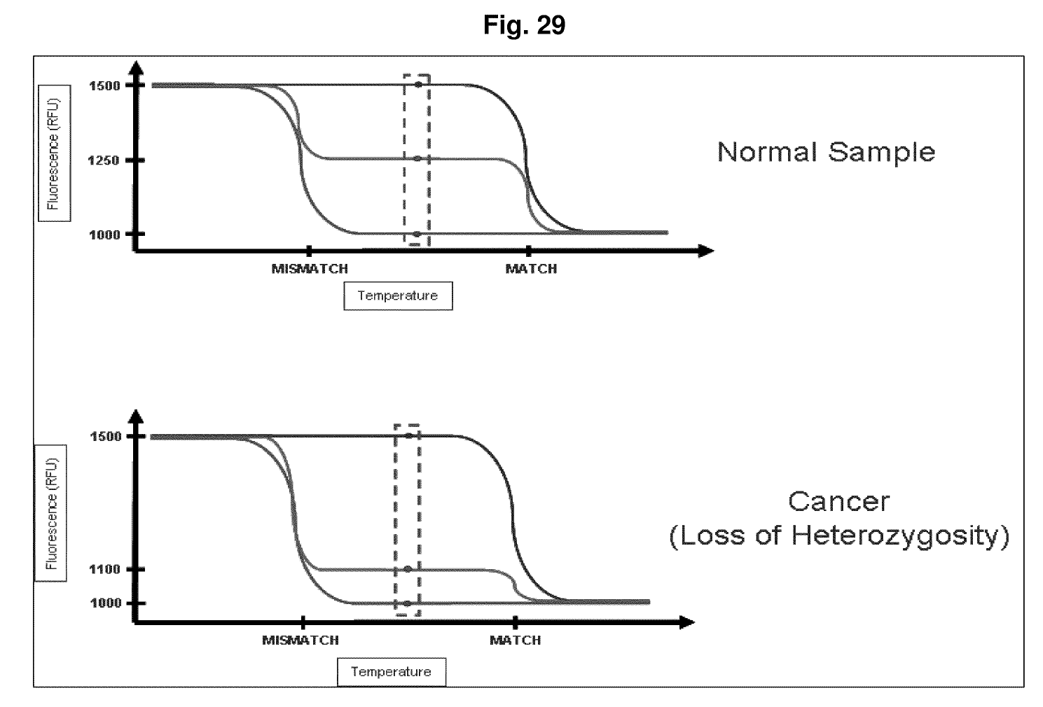

Similar to forensics and for many of the same reasons, dpFRET has the potential to advance both discovery and screening capabilities in clinical diagnostics and molecular pathology. Benefits are numerous for areas including cancer (FIG. 29), transplants, blood typing, pharmacogenetics and a host of other clinical fields.

Integration with Different Platforms—Portability and High Throughput. The dpFRET hybridization-based approach for both SNP and microsatellite genotyping has shown success with a number of different sample types for a number of different applications. One of the next steps for development of this novel technology for application to both forensic and clinical environments is incorporation into different platforms that provide either point-of-care or high throughput capabilities.

Lab-on-a-card microfluidic based systems have the ability to reproduce every step used in a laboratory setting with automated sample processing and analysis. In other words, microfluidic cards can extract, amplify and detect DNA sequences in an enclosed system which allows both portability of the system and minimal requirements for trained personnel. dpFRET has been successfully tested in just such a system designed by Micronics, Inc. (Seattle, Wash.). Implications for forensics and clinical diagnostics include on-site analysis of samples and point-of-care diagnosis of molecular based markers.

A number of high throughput PCR platforms have been developed in recent years. These include approaches from thermal cyclers capable of 384 reactions per run to unique platforms capable of >3000 reactions per run. Utilizing these platforms, dpFRET has potential for high-throughput for screening as well as discovery using ‘relative dpFRET sequencing’. Applications include genotyping changes in microbial and viral genomes, increased sample throughput for forensic laboratories and a host of other fields.

Having described the invention in detail, those skilled in the art will appreciate that modifications may be made of the invention without departing from the spirit and scope thereof. Therefore, it is not intended that the scope of the invention be limited to the specific embodiments described. Rather, it is intended that the appended claims and their equivalents determine the scope of the invention.

APPENDIX A

| Species | Sequence |
|---|---|
| Human | CAA CCG CCT TTT CAT CAA TCG CCC |
| | ACA TCA CTC GAG ACG TAA ATT ATG |
| | GC |
| Aardvark | CAA CCG CAT TCT CAT CTG TAA CCC |
| | ATA TTT GCC GAG ATG TAA ACT ACG |
| | GC |
| AfElephant | TAA CTG CAT TTT CAT CTA TAT CCC |
| | ATA TTT GCC GAG ATG TGA ACT ACG |
| | GC |
| Alpaca | CAA CAG CCT TCT CTT CAG TCG CAC |
| | ACA TCT GCC GAG ACG TAA ATT ACG |
| | GC |
| Armadillo | TAA CAG CCT TCT CAT CTG TAA CTC |
| | ACA TCT GCC GAG ACG TAA ACT ATG |
| | GC |
| AsBlkBear | CTA CAG CCT TTT CAT CAG TCG CCC |
| | ATA TTT GCC GAG ACG TCC ATT ACG |
| | GA |
| AsElephant | TAA CTG CAT TTT CAT CTA TAT CCC |
| | ATA TCT GCC GAG ACG TCA ACT ACG |
| | GC |
| AtWalrus | CCA CAG CTT TCT CAT CAA TCA CAC |
| | ATA TCT GCC GAG ATG TCA ACT ATG |
| | GT |
| AuSealLion | CCA CAG CCT TTT CAT CGG TCA CCC |
| | ACA TTT GCC GAG ACG TGA ACT ACG |
| | GC |
| Baboon | CCT CTG CCT TCT CTT CAA TCG CAC |
| | ACA TCA CCC GAG ACG TAA ACT ATG |
| | GC |
| BalWhale | CAA CCG CTT TCT CAT CAG TCA CAC |
| | ACA TTT GCC GAG ACG TAA ACT ACG |
| | GC |
| Bat | CTA CCG CAT TCA ACT CTG TCA CCC |
| | ATA TCT GTC GAG ACG TCA ACT ATG |
| | GA |
| BrnBear | CCA CAG CTT TTT CAT CAG TCA CCC |
| | ACA TTT GCC GAG ACG TTC ACT ACG |
| | GA |
| Buffalo | CAA CAG CAT TCT CCT CCG TCG CCC |
| | ACA TCT GCC GGG ACG TGA ACT ATG |
| | GA |
| CaspSeal | CCA CAG CCT TCT CAT CAG TAA CCC |
| | ACA TCT GCC GGG ACG TAA ACT ACG |
| | GC |
| Cat | TAA CCG CCT TTT CAT CAG TTA CCC |
| | ACA TCT GTC GCG ACG TTA ATT ATG |
| | GC |
| Catfish | CAA CTG CCT TTT CAT CCG TCG CCC |
| | ACA TCT GCC GAG ATG TAA ACT ACG |
| | GG |
| Cattle | CAA CAG CAT TCT CCT CTG TTA CCC |
| | ATA TCT GCC GAG ACG TGA ACT ACG |
| | GC |
| Cheetah | TAA CCG CCT TTT CAT CAG TTA CTC |
| | ACA TCT GCC GCG ACG TCA ACT ACG |
| | GC |
| Chicken | CCC TAG CCT TCT CCT CCG TAG CCC |
| | ACA CTT GCC GGA ACG TAC AAT ACG |
| | GC |
| Chimp | CAA CCG CCT TCT CAT CGA TCG CCC |
| | ACA TTA CCC GAG ACG TAA ACT ATG |
| | GT |
| Coelacanth | CAA CAG CAT TCT CAT CAG TAG CCC |
| | ACA TCT GCC GAG ATG TAA ACT ATG |
| | GA |
| Colobus | CCT CTG CTT TCT CCT CAG TTG CAC |
| | ATA TCA CCC GGG ACG TAA ACT ATG |
| | GC |
| Coyote | CCA CAG CTT TTT CAT CAG TCA CCC |
| | ACA TCT GTC GAG ACG TTA ACT ACG |
| | GC |
| Deer | TAA CAG CAT TCT CCT CTG TCA CCC |
| | ATA TCT GTC GAG ATG TCA ATT ATG |
| | GT |
| Desman | TAA CAG CCT TCT CAT CAG TAA CCC |
| | ATA TTT GCC GAG ATG TAA ACT ACG |
| | GA |
| Dog | CCA CAG CTT TTT CAT CAG TCA CCC |
| | ACA TCT GCC GAG ACG TTA ACT ACG |
| | GC |
| Dogfish | CCA CGG CCT TCT CCT CAG TAG TTC |
| | ATA TTT GTC GTG ACG TCA ATT ATG |
| | GT |
| Donkey | CAA CTG CCT TCT CAT CCG TCA CCC |
| | ATA TCT GCC GAG ACG TTA ACT ACG |
| | GA |
| Dugong | TAA CCG CAT TCT CCT CAG TAA CCC |
| | ATA TTT GCC GGG ATG TAA ACT ACG |
| | GC |
| Eel | CGA CCG CTT TCT CCT CAG TTG TCC |
| | ATA TCT GCC GAG ATG TAA ACT ATG |
| | GC |
| Finch | CCC TAG CCT TCT CCT CAG TCG CCC |
| | ACA TAT GCC GAG ACG TAC AAT TTG |
| | GC |
| FinWhale | CAA CCG CCT TCT CAT CAG TCA CAC |
| | ACA TCT GCC GAG ACG TGA ATT ACG |
| | GC |
| FlyFox | CAA CCG CCT TCC AAT CCG TAA CCC |
| | ACA TCT GCC GAG ACG TAA ACT ACG |
| | GC |
| Fox | CTA CTG CTT TCT CAT CTG TCA CTC |
| | ACA TCT GCC GAG ACG TTA ACT ATG |
| | GC |
| Frog | CCC TTG CAT TCT CAT CTA TTG CCC |
| | ACA TCT GTC GAG ATG TTA ATA ACG |
| | GC |
| GdFurSeal | CTA CAG CCT TTT CAT CAG TCA CCC |
| | ACA TTT GCC GAG ACG TGA ACT ACG |
| | GC |
| NtFurSeal | CCA CAG CCT TCT CAT CAG TCG CCC |
| | ATA TTT GCC GAG ACG TGA ACT ACG |
| | GC |
| Goat | TAA CAG CAT TTT CCT CTG TAA CTC |
| | ACA TTT GTC GAG ATG TAA ATT ATG |
| | GC |
| Goby | CCA CAG CTT TTT CTT CTG TAG CCC |
| | ATA TCT GCC GGG ATG TTA ACT TTG |
| | GT |
| Gorilla | CAA CCG CCT TCT CAT CAA TTG CCC |
| | ACA TCA CCC GAG ATG TAA ACT ATG |
| | GC |
| Gray Wolf | CCA CAG CTT TTT CAT CAG TCA CCC |
| | ACA TCT GCC GAG ACG TTA ACT ACG |
| | GC |
| Grebe | CCC TAG CCT TCT CAT CCG TCG CCC |
| | ACA CAT GTC GAA ACG TAC AGT ACG |
| | GC |
| GrnLizard | CCT CCG CAT TCT CAT CTG TCA CCC |
| | ACA TTC ACC GAG ATG TTC AAT ATG |
| | GC |
| GrnMonkey | CTT CTG CCT TCT CTT CAA TCG CAC |
| | ACA TCA CCC GAG ACG TAA ACC ACG |
| | GC |
| GuinPig | CCA CGG CAT TCT CGT CTG TCG CCC |
| | ACA TTT GCC GAG ACG TAA ACT ATG |
| | GC |
| Hamster | CTA CAG CAT TCT CAT CAG TCA CCC |
| | ACA TTT GTC GAG ATG TTA ATT ACG |
| | GC |
| Hedgehog | TTA CAG CAT TTT CAT CCA TTA CTC |
| | ACA TTT GCC GAG ATG TAA ACT ACG |
| | GT |
| Heron | CAT TAG CCT TCT CAT CCG TCG CCC |
| | ACA CAT GCC GAA ACG TAC AGT ACG |
| | GC |
| Hippo | TCA CCG CAT TCT CAT CGG TAA CCC |
| | ACA TCT GCC GTG ATG TAA ACT ACG |
| | GG |
| Horse | CAA CTG CCT TCT CAT CCG TCA CTC |
| | ACA TCT GCC GAG ACG TTA ACT ACG |
| | GA |
| HumWhale | CAA CCG CCT TCT CAT CAG TCA CAC |
| | ACA TCT GTC GAG ACG TAA ATT ATG |
| | GC |
| Hyrax | TAA CCG CAT TCA CAT CAG TAA CCC |
| | ACA TTT GTC GAG ACG TAA ACC ATG |
| | GA |
| Junglefowl | CCC TAG CCT TCT CCT CCG TAG CCC |
| | ACA CTT GCC GGA ACG TAC AAT ACG |
| | GC |
| Kestrel | CAC TGG CCT TCT CAT CTG TTG CCC |
| | ACA CAT GCC GAA ACG TGC AGT ACG |
| | GA |
| Kiwi | CCC TAG CCT TTT CAT CCA TCG CCC |
| | ATA TCT GTC GAA ACG TCC AAT ATG |
| | GA |
| Langur | CCT CAG CCT TCT CCT CAA TCG CCC |
| | ATA TCA CTC GAG ACG TAA ACT ACG |
| | GC |
| Lemur | CAA CAG CAT TTT CAT CCA TTG CCC |
| | ACA TCT CAC GAG ACG TAA ACT ACG |
| | GC |
| Leopard | TAA CTG CTT TCT CAT CTG TCA CCC |
| | ATA TTT GCC GCG ACG TAA ACT ATG |
| | GT |
| LfMonkey | CCT CTG CCT TCT CCT CAA TTG CAC |
| | ATA TTA CCC GAG ATG TAA ATT ATG |
| | GC |
| Loach | CTA CTG CCT TTT CAT CCG TAG CCC |
| | ACA TCT GCC GAG ATG TTA ACT ATG |
| | GA |
| Loon | CCC TAG CCT TCT CAT CCG TTG CCC |
| | ACA CAT GCC GAA ACG TAC AGT ACG |
| | GT |
| LprdSeal | CTA CAG CCT TTT CAT CAG TCA CAC |
| | ACA TCT GCC GAG ACG TAA ACT ACG |
| | GT |
| Mammoth | TAA CTG CAT TTT CAT CTA TAT CCC |
| | ATA TCT GCC GAG ATG TCA ACT ACG |
| | GT |
| Minnow | CCA CTG CAT TTT CAT CAG TAG CCC |
| | ACA TCT GCC GAG ATG TTA ATT ATG |
| | GC |
| MnkSeal | CCA CAG CCT TTT CAT CAA TCA CAC |
| | ACA TCT GCC GAG ACG TAA ATT ACG |
| | GC |
| Mongoose | CAA CTG CCT TTT CAT CAG TAA CCC |
| | ACA TTT GCC GCG ACG TCA ACT ACG |
| | GC |
| Mouse | TAA CAG CCT TTT CAT CAG TAA CAC |
| | ACA TTT GTC GAG ACG TAA ATT ACG |
| | GG |
| Muntjac | TAA CAG CAT TCT CCT CGG TTA CCC |
| | ATA TCT GCC GAG ACG TCA ACT ATG |
| | GC |
| NileCroc | CCC TAG CTT TTA TAT CTG TCG CTT |
| | ATA CTT CAC GAG AAG TTT GAT ACG |
| | GC |
| Orangutan | CCA CTG CCT TTT CAT CAA TCG CCC |
| | ACA TCA CTC GAG ATG TAA ACT ACG |
| | GC |
| Penguin | CCC TAG CCT TCT CCT CCA TCG CCC |
| | ACA CAT GCC GAA ATG TAC AGT ACG |
| | GC |
| Pig | CAA CAG CTT TCT CAT CAG TTA CAC |
| | ACA TTT GTC GAG ACG TAA ATT ACG |
| | GA |
| PolarBear | CCA CAG CTT TTT CAT CAG TCA CCC |
| | ACA TTT GCC GAG ACG TTC ACT ACG |
| | GG |
| Porpoise | CAA CCG CTT TTT CAT CAG TCG CAC |
| | ATA TCT GTC GAG ACG TTA ATT ATG |
| | GC |
| Rabbit | CAA CAG CAT TCT CAT CAG TAA CCC |
| | ATA TTT GCC GAG ATG TTA ACT ATG |
| | GC |
| Rat | TAA CAG CAT TTT CAT CAG TCA CCC |
| | ACA TCT GCC GAG ACG TAA ACT ACG |
| | GC |
| Reindeer | TAA CAG CAT TCT CCT CTG TTA CTC |
| | ACA TCT GTC GAG ACG TCA ATT ATG |
| | GC |
| RghtWhale | CAA CCG CCT TCT CAT CAA TCA CAC |
| | ACA TCT GTC GAG ACG TAA ACT ACG |
| | GT |
| Rhea | CAT TAG CCT TCT CAT CCG TAG CCC |
| | ACA CCT GCC GCA ACG TCC AAT ATG |
| | GT |
| Rhino | TAA CTG CCT TCT CAT CTG TCG CCC |
| | ATA TCT GTC GAG ACG TGA ATT ACG |
| | GC |
| RvrDolphin | CAA CCG CCT TCT CAT CCA TCA CAC |
| | ACA TTT GCC GAG ACG TCA ACT ACG |
| | GC |
| Salamander | CTT CCG CAT TTT CAT CAG TCG TAC |
| | ATA TCT GCC GAG ACG TAA ACT ATG |
| | GA |
| Salmon | CAA CAG CTT TTT CCT CTG TCT GCC |
| | ACA TCT GCC GAG ATG TTA GTT ACG |
| | GC |
| Sheep | CAA CAG CAT TCT CCT CTG TAA CCC |
| | ACA TTT GCC GAG ACG TAA ACT ATG |
| | GC |
| Skate | CCT CCG CTT TCT CCT CAG TTG TTC |
| | ACA TCT GCC GAG ATG TGA ATT ATG |
| | GA |
| Sloth | CCA CCG CCT TCT CAT CCG TAA CCC |
| | ACA TCT GCC GAG ACG TAA ACT ACG |
| | GC |
| SptSeal | CCA CAG CCT TCT CAT CAG TAA CCC |
| | ACA TCT GCC GAG ACG TAA ACT ACG |
| | GC |
| Squirrel | TAA CAG CTT TTT CTT CCG TTA CTC |
| | ACA TCT GCC GAG ACG TAA ATT ATG |
| | GC |
| Stingray | CAA CCG CAT TCT CCT CAG TAG CAC |
| | ATA TCT GCC GAG ACG TAA ACT ACG |
| | GC |
| Sturgeon | CAA CAG CCT TCT CTT CTG TCG CCC |
| | ACA TCT GCC GAG ATG TAA ATT ACG |
| | GA |
| TftDeer | TAA CAG CAT TTT CCT CTG TAA CCC |
| | ACA TTT GCC GAG ACG TCA ACT ATG |
| | GG |
| TwnVole | CAA CAG CAT TCT CAT CAG TAG CCC |
| | ATA TCT GCC GAG ACG TCA ACT ACG |
| | GC |
| Vole | CAA CAG CAT TCT CAT CAG TAG CCC |
| | ACA TTT GTC GAG ACG TAA ACT ATG |
| | GC |
| WhtShark | CTA TAG CCT TCT CCT CAG TAA CCC |
| | ACA TCT GCC GTG ACG TCA ATT ACG |
| | GC |
| Yak | CAA CAG CAT TCT CCT CCG TTG CCC |
| | ATA TCT GCC GAG ACG TGA ACT ACG |
| | GC |
| Label | Sequence |
| CytB_Reference | CAACCGCCTTTTCATCAATCGCCCACATCACT |
| | CGAGACGTAAATTATGGC |
| CytB_1A | CAACCGCCTTATCATCAATCGCCCACATCACT |
| | CGAGACGTAAATTATGGC |
| CytB_1C | CAACCGCCTTCTCATCAATCGCCCACATCACT |
| | CGAGACGTAAATTATGGC |
| CytB_1G | CAACCGCCTTGTCATCAATCGCCCACATCACT |
| | CGAGACGTAAATTATGGC |
| CytB_2A | CAACCGCCTTTACATCAATCGCCCACATCACT |
| | CGAGACGTAAATTATGGC |
| CytB_2C | CAACCGCCTTTCCATCAATCGCCCACATCACT |
| | CGAGACGTAAATTATGGC |
| CytB_2G | CAACCGCCTTTGCATCAATCGCCCACATCACT |
| | CGAGACGTAAATTATGGC |
| CytB_3T | CAACCGCCTTTTTATCAATCGCCCACATCACT |
| | CGAGACGTAAATTATGGC |
| CytB_3A | CAACCGCCTTTTAATCAATCGCCCACATCACT |
| | CGAGACGTAAATTATGGC |
| CytB_3G | CAACCGCCTTTTGATCAATCGCCCACATCACT |
| | CGAGACGTAAATTATGGC |
| CytB_4T | CAACCGCCTTTTCTTCAATCGCCCACATCACT |
| | CGAGACGTAAATTATGGC |
| CytB_4C | CAACCGCCTTTTCCTCAATCGCCCACATCACT |
| | CGAGACGTAAATTATGGC |
| CytB_4G | CAACCGCCTTTTCGTCAATCGCCCACATCACT |
| | CGAGACGTAAATTATGGC |
| CytB_5A | CAACCGCCTTTTCAACAATCGCCCACATCACT |
| | CGAGACGTAAATTATGGC |
| CytB_5C | CAACCGCCTTTTCACCAATCGCCCACATCACT |
| | CGAGACGTAAATTATGGC |
| CytB_5G | CAACCGCCTTTTCAGCAATCGCCCACATCACT |
| | CGAGACGTAAATTATGGC |
| CytB_6T | CAACCGCCTTTTCATTAATCGCCCACATCACT |
| | CGAGACGTAAATTATGGC |
| CytB_6A | CAACCGCCTTTTCATAAATCGCCCACATCACT |
| | CGAGACGTAAATTATGGC |
| CytB_6G | CAACCGCCTTTTCATGAATCGCCCACATCACT |
| | CGAGACGTAAATTATGGC |
| CytB_7T | CAACCGCCTTTTCATCTATCGCCCACATCACT |
| | CGAGACGTAAATTATGGC |
| CytB_7C | CAACCGCCTTTTCATCCATCGCCCACATCACT |
| | CGAGACGTAAATTATGGC |
| CytB_7G | CAACCGCCTTTTCATCGATCGCCCACATCACT |
| | CGAGACGTAAATTATGGC |
| CytB_8T | CAACCGCCTTTTCATCATCGCCCACATCACTC |
| | GAGACGTAAATTATGGC |
| CytB_8C | CAACCGCCTTTTCATCACTCGCCCACATCACT |
| | CGAGACGTAAATTATGGC |
| CytB_8G | CAACCGCCTTTTCATCAGTCGCCCACATCACT |
| | GAGACGTAAATTATGGC |
| CytB_9A | CAACCGCCTTTTCATCAAACGCCCACATCACT |
| | CGAGACGTAAATTATGGC |
| CytB_9C | CAACCGCCTTTTCATCAACCGCCCACATCACT |
| | CGAGACGTAAATTATGGC |
| CytB_9G | CAACCGCCTTTTCATCAAGCGCCCACATCACT |
| | CGAGACGTAAATTATGGC |
| CytB_10T | CAACCGCCTTTTCATCAATTGCCCACATCACT |
| | CGAGACGTAAATTATGGC |
| CytB_10A | CAACCGCCTTTTCATCAATAGCCCACATCACT |
| | CGAGACGTAAATTATGGC |
| CytB_10G | CAACCGCCTTTTCATCAATGGCCCACATCACT |
| | CGAGACGTAAATTATGGC |
| CytB_11C | CAACCGCCTTTTCATCAATCCCCCACATCACT |
| | CGAGACGTAAATTATGGC |
| CytB_11T | CAACCGCCTTTTCATCAATCTCCCACATCACT |
| | CGAGACGTAAATTATGGC |
| CytB_11A | CAACCGCCTTTTCATCAATCACCCACATCACT |
| | CGAGACGTAAATTATGGC |
| CytB_12T | CAACCGCCTTTTCATCAATCGTCCACATCACT |
| | CGAGACGTAAATTATGGC |
| CytB_12A | CAACCGCCTTTTCATCAATCGACCACATCACT |
| | CGAGACGTAAATTATGGC |
| CytB_12G | CAACCGCCTTTTCATCAATCGGCCACATCACT |
| | CGAGACGTAAATTATGGC |
| CytB_13T | CAACCGCCTTTTCATCAATCGCTCACATCACT |
| | CGAGACGTAAATTATGGC |
| CytB_13A | CAACCGCCTTTTCATCAATCGCACACATCACT |
| | CGAGACGTAAATTATGGC |
| CytB_13G | CAACCGCCTTTTCATCAATCGCGCACATCACT |
| | CGAGACGTAAATTATGGC |
| CytB_14T | CAACCGCCTTTTCATCAATCGCCTACATCACT |
| | CGAGACGTAAATTATGGC |
| CytB_14A | CAACCGCCTTTTCATCAATCGCCAACATCACT |
| | CGAGACGTAAATTATGGC |
| CytB_14G | CAACCGCCTTTTCATCAATCGCCGACATCACT |
| | CGAGACGTAAATTATGGC |
| CytB_15T | CAACCGCCTTTTCATCAATCGCCCTCATCACT |
| | CGAGACGTAAATTATGGC |
| CytB_15C | CAACCGCCTTTTCATCAATCGCCCCCATCACT |
| | CGAGACGTAAATTATGGC |
| CytB_15G | CAACCGCCTTTTCATCAATCGCCCGCATCACT |
| | CGAGACGTAAATTATGGC |
| CytB_16T | CAACCGCCTTTTCATCAATCGCCCATATCACT |
| | CGAGACGTAAATTATGGC |
| CytB_16A | CAACCGCCTTTTCATCAATCGCCCAAATCACT |
| | CGAGACGTAAATTATGGC |
| CytB_16G | CAACCGCCTTTTCATCAATCGCCCAGATCACT |
| | CGAGACGTAAATTATGGC |
| CytB_17T | CAACCGCCTTTTCATCAATCGCCCACTTCACT |
| | CGAGACGTAAATTATGGC |
| CytB_17C | CAACCGCCTTTTCATCAATCGCCCACCTCACT |
| | CGAGACGTAAATTATGGC |
| CytB_17G | CAACCGCCTTTTCATCAATCGCCCACGTCACT |
| | CGAGACGTAAATTATGGC |
| CytB_18A | CAACCGCCTTTTCATCAATCGCCCACAACACT |
| | CGAGACGTAAATTATGGC |
| CytB_18C | CAACCGCCTTTTCATCAATCGCCCACACCACT |
| | CGAGACGTAAATTATGGC |
| CytB_18G | CAACCGCCTTTTCATCAATCGCCCACAGCACT |
| | CGAGACGTAAATTATGGC |
| CytB_19T | CAACCGCCTTTTCATCAATCGCCCACATTACT |
| | CGAGACGTAAATTATGGC |
| CytB_19A | CAACCGCCTTTTCATCAATCGCCCACATAACT |
| | CGAGACGTAAATTATGGC |
| CytB_19G | CAACCGCCTTTTCATCAATCGCCCACATGACT |
| | CGAGACGTAAATTATGGC |
| CytB_20T | CAACCGCCTTTTCATCAATCGCCCACATCTCT |
| | CGAGACGTAAATTATGGC |
| CytB_20C | CAACCGCCTTTTCATCAATCGCCCACATCCCT |
| | CGAGACGTAAATTATGGC |
| CytB_20G | CAACCGCCTTTTCATCAATCGCCCACATCGCT |
| | CGAGACGTAAATTATGGC |
| CytB_21T | CAACCGCCTTTTCATCAATCGCCCACATCATT |
| | CGAGACGTAAATTATGGC |
| CytB_21A | CAACCGCCTTTTCATCAATCGCCCACATCAAT |
| | CGAGACGTAAATTATGGC |
| CytB_21G | CAACCGCCTTTTCATCAATCGCCCACATCAGT |
| | CGAGACGTAAATTATGGC |
| CytB_22A | CAACCGCCTTTTCATCAATCGCCCACATCACA |
| | CGAGACGTAAATTATGGC |
| CytB_22C | CAACCGCCTTTTCATCAATCGCCCACATCACC |
| | CGAGACGTAAATTATGGC |
| CytB_22G | CAACCGCCTTTTCATCAATCGCCCACATCACG |
| | CGAGACGTAAATTATGGC |
| CytB_23T | CAACCGCCTTTTCATCAATCGCCCACATCACT |
| | TGAGACGTAAATTATGGC |
| CytB_23A | CAACCGCCTTTTCATCAATCGCCCACATCACT |
| | AGAGACGTAAATTATGGC |
| CytB_23G | CAACCGCCTTTTCATCAATCGCCCACATCACT |
| | GGAGACGTAAATTATGGC |
| CytB_24T | CAACCGCCTTTTCATCAATCGCCCACATCACT |
| | CTAGACGTAAATTATGGC |
| CytB_24A | CAACCGCCTTTTCATCAATCGCCCACATCACT |
| | CAAGACGTAAATTATGGC |
| CytB_24C | CAACCGCCTTTTCATCAATCGCCCACATCACT |
| | CCAGACGTAAATTATGGC |
| CytB_25T | CAACCGCCTTTTCATCAATCGCCCACATCACT |
| | CGTGACGTAAATTATGGC |
| CytB_25C | CAACCGCCTTTTCATCAATCGCCCACATCACT |
| | CGCGACGTAAATTATGGC |
| CytB_25G | CAACCGCCTTTTCATCAATCGCCCACATCACT |
| | CGGGACGTAAATTATGGC |
| CytB_26T | CAACCGCCTTTTCATCAATCGCCCACATCACT |
| | CGATACGTAAATTATGGC |
| CytB_26A | CAACCGCCTTTTCATCAATCGCCCACATCACT |
| | CGAAACGTAAATTATGGC |
| CytB_26C | CAACCGCCTTTTCATCAATCGCCCACATCACT |
| | CGACACGTAAATTATGGC |
| CytB_27T | CAACCGCCTTTTCATCAATCGCCCACATCACT |
| | CGAGTCGTAAATTATGGC |
| CytB_27C | CAACCGCCTTTTCATCAATCGCCCACATCACT |
| | CGAGCCGTAAATTATGGC |
| CytB_27G | CAACCGCCTTTTCATCAATCGCCCACATCACT |
| | CGAGGCGTAAATTATGGC |
| CytB_28T | CAACCGCCTTTTCATCAATCGCCCACATCACT |
| | CGAGATGTAAATTATGGC |
| CytB_28A | CAACCGCCTTTTCATCAATCGCCCACATCACT |
| | CGAGAAGTAAATTATGGC |
| CytB_28G | CAACCGCCTTTTCATCAATCGCCCACATCACT |
| | CGAGAGGTAAATTATGGC |
| CytB_29T | CAACCGCCTTTTCATCAATCGCCCACATCACT |
| | CGAGACTTAAATTATGGC |
| CytB_29A | CAACCGCCTTTTCATCAATCGCCCACATCACT |
| | CGAGACATAAATTATGGC |
| CytB_29C | CAACCGCCTTTTCATCAATCGCCCACATCACT |
| | CGAGACCTAAATTATGGC |
| CytB_30A | CAACCGCCTTTTCATCAATCGCCCACATCACT |
| | CGAGACGAAAATTATGGC |
| CytB_30C | CAACCGCCTTTTCATCAATCGCCCACATCACT |
| | CGAGACGCAAATTATGGC |
| CytB_30G | CAACCGCCTTTTCATCAATCGCCCACATCACT |
| | CGAGACGGAAATTATGGC |
| CytB_27T28T29T30A | CAACCGCCTTTTCATCAATCGCCCACATCACT |
| | CGAGTTTAAAATTATGGC |
| CytB_1A2A3A4T | CAACCGCCTTAAATTCAATCGCCCACATCACT |
| | CGAGACGTAAATTATGGC |
| CytB_5A6A7T8T | CAACCGCCTTTTCAAATTTCGCCCACATCACT |
| | CGAGACGTAAATTATGGC |
| CytB_22A23A24A25T | CAACCGCCTTTTCATCAATCGCCCACATCACA |
| | AATGACGTAAATTATGGC |
| REP_ATTTTA | CAACCGCCTTATTTTAATTTTAATTTTAATTT |
| | TAATTTTAAAATTATGGC |

1. A method for identifying at least one single nucleotide polymorphism in a target nucleic acid sequence comprising the steps of:
generating a single-stranded target nucleic acid sequence;
adding a donor intercalating dye and a complementary fluoropohore labeled probe to said target nucleic acid sequence to form a probe/target hybrid with dye deposited between the probe and target;
hybridizing said combined sample and exposing to a specific wavelength of light;
monitoring FRET fluorescent emmision from said excited combined sample associated with one or both of the hybridization of a universal sequence to said target sequence and the dissociation of said universal sequence from said target sequence;
analyzing said combined sample using a melt-curve analysis to identify at least one single nucleotide polymorphism therein; and
discriminating differences across any sequence distance using at least one probe.

2. The method of claim 1 wherein said target nucleic acid sequence is derived from a DNA source selected from the group consisting of fungal, plant, yeast, bacterial, viral, human, animal, any other living organism, and combinations thereof.

3. The method of claim 1 wherein said donor intercalating dye is SYBR Green I.

4. The method of claim 1 wherein the fluorophore in said fluorophore labeled probe is Texas Red.

5. The method of claim 1 wherein said single-stranded nucleic acid sequence is generated using asymmetric PCR.

6. A method for identifying at least one microsatellite in a target nucleic acid sequence comprising the steps of:
generating a single-stranded target nucleic acid sequence;
adding a donor intercalating dye and a complementary fluoropohore labeled allele specific probe to said target nucleic acid sequence to form a probe/target hybrid with dye deposited between the probe and target;
hybridizing said combined sample;
exposing said combined sample to a specific wavelength of light;
monitoring FRET fluorescent emmision from said excited combined sample associated with one or both of the hybridization of a universal sequence to said target sequence and the dissociation of said universal sequence from said target sequence;
analyzing said combined sample using a melt-curve analysis to identify at least one microsatellite therein; and
discriminating differences across any sequence distance using at least one probe.

7. The method of claim 6 wherein said target nucleic acid sequence is derived from a DNA source selected from the group consisting of fungal, plant, yeast, bacterial, viral, human, animal, any other living organism, and combinations thereof.

8. The method of claim 6 wherein said donor intercalating dye is SYBR Green I.

9. The method of claim 6 wherein the fluorophore in said fluorophore labeled probe is Texas Red.

10. The method of claim 6 wherein said single-stranded nucleic acid sequence is generated using asymmetric PCR.

11. A method for identifying at least one insertion/deletion loci in a target nucleic acid sequence comprising the steps of:
generating a single-stranded target nucleic acid sequence;
adding a donor intercalating dye and a complementary fluoropohore labeled probe to said target nucleic acid sequence to form a probe/target hybrid with dye deposited between the probe and target;
hybridizing said combined sample and exposing to a specific wavelength of light;
monitoring FRET fluorescent emmision from said excited combined sample associated with one or both of the hybridization of a universal sequence to said target sequence and the dissociation of said universal sequence from said target sequence;
analyzing said combined sample using a melt-curve analysis to identify at least one single nucleotide polymorphism therein; and
discriminating differences across any sequence distance using at least one probe.

12. The method of claim 11 wherein said target nucleic acid sequence is derived from a DNA source selected from the group consisting of fungal, plant, yeast, bacterial, viral, human, animal, any other living organism, and combinations thereof.

13. The method of claim 11 wherein said donor intercalating dye is SYBR Green I.

14. The method of claim 11 wherein the fluorophore in said fluorophore labeled probe is Texas Red.

15. The method of claim 11 wherein said single-stranded nucleic acid sequence is generated using asymmetric PCR.
